(19) United States
(12) Patent Application Publication
(10) Pub. No.: US 2024/0317917 A1
(43) Pub. Date: Sep. 26, 2024

(54) METHODS FOR HYDROAMINOALKYLATION OF OLEFINS AND GROUP 4 METAL COMPLEXES USEFUL IN SUCH METHODS

(71) Applicant: THE UNIVERSITY OF BRITISH COLUMBIA, Vancouver, British Columbia (CA)

(72) Inventors: Laurel L. SCHAFER, Vancouver (CA); Manfred MANSSEN, Vancouver (CA); Danfeng DENG, Vancouver (CA); Rebecca DIPUCCHIO, Vancouver (CA); Sabrina SCOTT, Vancouver (CA)

(21) Appl. No.: 18/268,464

(22) PCT Filed: Dec. 21, 2021
(86) PCT No.: PCT/CA21/51857

§ 371 Date: Jun. 20, 2023

Related U.S. Application Data
(60) Provisional application No. 63/130,306, filed on Dec. 23, 2020.

(51) Int. Cl. C08F 255/08; C08F 2/12; C08F 2/36; C08F 4/16

(52) U.S. Cl. CPC C08F 255/08; C08F 2/12; C08F 2/36; C08F 4/16

(57) ABSTRACT

The present disclosure relates to methods for the hydroaminoalkylation of an olefin. Such methods can comprise reacting the olefin with a secondary amine in the presence of a catalyst of Formula (I). The present disclosure also relates to catalysts which can be useful in such methods. LₓM(R₁)_{y}(I)

### CROSS-REFERENCE TO RELATED APPLICATIONS

The present application claims the benefit of priority from co-pending U.S. provisional application No. 63/130,306 filed on Dec. 23, 2020, the contents of which are incorporated herein by reference in their entirety.

### FIELD

The present disclosure relates to methods for the hydroaminoalkylation of an olefin as well as group 4 metal complexes which can be used, for example, as a catalyst in such methods.

### BACKGROUND

Currently, the most commonly used concept for amine synthesis in industry is the hydroformylation of alkenes, which uses primarily cobalt- or rhodium-containing hydroformylation catalysts, and subsequent reductive amination.¹

Hydroaminoalkylation is an emerging reaction that is an atom-economic catalytic reaction forming new C_{sp′}-C_{sp′} bonds by hydrofunctionalization of C—C multiple bonds with a C—H bond α- to the nitrogen of an amine and avoids pre-functionalized coupling partners.² Consequently, readily available amine and alkene feedstocks can, for example, be used. The efficient synthesis of amine products may, for example, be useful for generating small molecules relevant to industries such as the pharmaceutical, agrochemical and/or fine chemical industries.³

Hydroaminoalkylation typically utilizes less toxic and less expensive earth-abundant base metals than hydroformylation; mainly the early-transition group 4 and 5 metals (ETMs). Since first being reported by Clerici and Maspero in 1980⁴ and subsequently by Herzon and Hartwig in 2007⁵, the scope of hydroaminoalkylation has been expanded and several active catalytic systems, primarily for the elements tantalum and titanium, have been reported.^{2,6} The use of titanium for this application may be desirable, for example as it is the second most abundant transition metal⁷ and it is already used in certain industrial processes.⁸

Relatively inactive homoleptic systems (e.g. Ti(NMe₂)₄,⁹ TiBn₄,¹⁰ Ta(NMe₂)₅¹¹) and highly electronically stabilizing indenyl ligands,¹² have been reported for titanium and tantalum for use in hydroaminoalkylation as well as certain 1,3-N,X-chelated catalyst systems (X=N or O). For example, for tantalum, 1,3-N,O-chelating ligands, which were first introduced in the form of pyridonate, amidate and ureate complexes for group 4 catalyzed hydroamination, have been utilized pre-isolated or in situ.¹³ Secondary amine-containing polymers have also been prepared using tantalum catalyzed hydroaminoalkylation of dienes and subsequent ring-opening metathesis polymerization (ROMP).¹⁴ However, it remains desirable to have a catalyst that can both deliver useful turnover frequencies (TOFs) and turnover numbers (TONs) for the reaction of amines such as N-methylaniline derivates with either terminal or challenging internal alkenes as well as be useful to catalyze hydroaminoalkylation using alkyl amine substrates.

In the case of titanium, 1,3-N,N-chelating ligands in the form of primarily disubstituted aminopyridinate catalysts and mono-coordinated formamidinate ligands (pre-isolated and in situ), were first reported by Eisen for the polymerization of propylene.¹⁵ The aminopyridinate displayed activity for intramolecular hydroaminoalkylation¹⁶ and improved catalyst-controlled selectivity for the linear product¹⁷ and can transform industrial relevant feedstocks like dimethylamine¹⁸ and ethylene.¹⁹ In general, these titanium catalysts need relatively harsh reactions conditions (up to 180° C. in n-hexane) with reaction times up to 96 h. The in situ use of mono-coordinated formamidinate ligands with TiBn₄ as the titanium source and solvent-free conditions were used to yield a catalyst system that can result in reduced reaction times from days to minutes.²⁰ However, the multi-step laborious lengthy synthesis of the ligand and the use of the expensive and light- and temperature-sensitive TiBn₄ (typically requires storage at −30° C. in a glovebox), are disadvantages of this catalyst approach.

### SUMMARY

An earth-abundant titanium catalyst can be readily synthesized, for example by using commercially available and inexpensive Ti(NMe₂)₄ and a ureate or amidate ligand in situ. For example, the system using a sterically demanding ureate ligand exhibited exceptional turnover frequencies (TOFs) in comparison to reported titanium catalysts based upon Ti(NMe₂)₄ for a wide range of hydroaminoalkylation substrates and can even complete reactions with challenging substrates such as dialkylamines within a day. Furthermore, such catalysts may allow, for example, for new handling methods for hydroaminoalkylation without the necessity of glovebox techniques and a wide-ranging practical applicability in the “green” synthesis, for example, of amine-functionalized materials, amino acids and N-heterocyclic building blocks for medicinal chemistry.

Accordingly, the present disclosure includes a method for the hydroaminoalkylation of an olefin, the method comprising reacting the olefin with a secondary amine in the presence of a catalyst of Formula (I):

LₓM(R¹)_{y}(I),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L is a ligand of Formula (II)

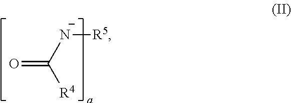

wherein
each a is independently 1 or 2;
each R⁴ is independently C₁₋₄₀alkyl, C₃₋₄₀cycloalkyl, substituted or unsubstituted aryl or NR⁶R⁷;
   (i) when a is 1,
      R⁵ is C₁₋₄₀alkyl, C₃₋₄₀cycloalkyl or substituted or unsubstituted aryl; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and R⁶ is C₁₋₄₀alkyl, C₃₋₄₀cycloalkyl, substituted or unsubstituted aryl or C₁₋₁₀alkylene-aryl, wherein the aryl is substituted or unsubstituted;
   (ii) when a is 2,
      R⁵ is C₁₋₄₀alkylene, C₃₋₄₀cycloalkylene or substituted or unsubstituted arylene; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

In an embodiment, a is 1.

In an embodiment, x is 1 or 2. In another embodiment, x is 1.

In an embodiment, R⁵ is C₁₋₁₆alkyl, C₃₋₁₆cycloalkyl or substituted aryl. In another embodiment, R⁵ is substituted aryl. In a further embodiment, R⁵ is 2,6-diisopropylphenyl.

In an embodiment, R⁴ is NR⁶R⁷, and R⁶ and R⁷ are each independently C₁₋₁₆alkyl, C₃₋₁₆cycloalkyl, substituted or unsubstituted aryl or C₁₋₆alkylene-aryl.

In an embodiment, R⁶ and R⁷ are different.

In an embodiment, R⁶ is C₁₋₆alkyl and R⁷ is C₁₋₄alkylene-aryl. In another embodiment, R⁶ is methyl and R⁷ is —CH(CH₃)-phenyl.

In an embodiment, R⁴ is C₁₋₁₀alkyl or unsubstituted aryl. In another embodiment, R⁴ is tert-butyl or phenyl.

In an embodiment, R¹ is NMe₂ or CH₂Si(CH₃)₃. In another embodiment, R¹ is NMe₂.

In an embodiment, M is Ti.

In an embodiment, the catalyst is prepared from the reaction of a compound of Formula (III) with a group 4 metal complex of Formula (IV):

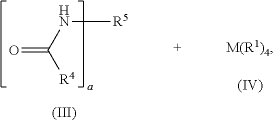

wherein M, R¹, R⁴, R⁵ and a are as defined in the present disclosure.

In an embodiment, the catalyst is generated in situ.

In an embodiment, the reaction of the olefin with the secondary amine is carried out without addition of solvent.

In an embodiment, the olefin comprises a terminal alkene. In another embodiment, the olefin comprises a silyl protected alcohol. In an embodiment, the olefin comprises a cyclic alkene. In another embodiment, the reaction of the olefin with the secondary amine produces an amine-substituted monomer for ring-opening metathesis polymerization (ROMP). In an embodiment, the olefin is a polymer comprising an alkene. In a further embodiment, the polymer is an alkene-terminated polyolefin.

In an embodiment, the secondary amine is of the formula NR^{a}R^{b}, wherein R^{a} and R^{b} are each independently substituted or unsubstituted C₁₋₄₀alkyl, C₃₋₄₀cycloalkyl, C₁₋₁₀alkylene-C₃₋₄₀cycloalkyl, aryl or C₁₋₁₀alkylene-aryl or R^{a} and R^{b} are bonded together, thereby forming, together with the nitrogen atom they are both bound to, a substituted or unsubstituted heterocycle that is optionally part of a condensed ring system.

In an embodiment, R^{a} is C₁₋₄alkyl and R^{b} is substituted or unsubstituted aryl.

In an embodiment, the amine is of the formula

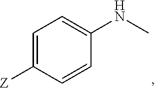

wherein Z is H, halo, —O—C₁₋₁₀alkyl, —O—C₃₋₁₀cycloalkyl, C₁₋₁₀alkyl or C₃₋₁₀cycloalkyl.

The present disclosure also includes a compound of Formula I(a):

(L^{a})ₓM(R¹)_{y}I(a),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L^{a} is a ligand of Formula II(a)

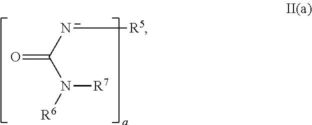

wherein
each a is independently 1 or 2;
   (i) when a is 1,
      R⁵ is C₁₋₄₀alkyl, C₃₋₄₀cycloalkyl or substituted or unsubstituted aryl; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and R⁶ is C₁₋₄₀alkyl, C₃₋₄₀cycloalkyl, substituted or unsubstituted aryl or C₁₋₁₀alkylene-aryl, wherein the aryl is substituted or unsubstituted;
   (ii) when a is 2,
      R⁵ is C₁₋₄₀alkylene, C₃₋₄₀cycloalkylene or substituted or unsubstituted arylene; and R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

In an embodiment, a is 1.

In an embodiment, x is 1 or 2. In an embodiment, x is 1.

In an embodiment, R⁵ is C₁₋₁₆alkyl, C₃₋₁₆cycloalkyl or substituted aryl. In another embodiment, R⁵ is substituted aryl. In a further embodiment, R⁵ is 2,6-diisopropylphenyl.

In an embodiment, R⁶ and R⁷ are each independently C₁₋₁₆alkyl, C₃₋₁₆cycloalkyl, substituted or unsubstituted aryl or C₁₋₆alkylene-aryl.

In an embodiment, R⁶ and R⁷ are different.

In an embodiment, R⁶ is C₁₋₆alkyl and R⁷ is C₁₋₄alkylene-aryl. In another embodiment, R⁶ is methyl and R⁷ is —CH(CH₃)-phenyl.

In an embodiment, R¹ is NMe₂ or CH₂Si(CH₃)₃. In another embodiment, R¹ is NMe₂.

In an embodiment, M is Ti

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows an Oak Ridge thermal-ellipsoid plot program (ORTEP) representation of the molecular structure of bis(3-(2,6-diisopropylphenyl)-1-methyl-1-(1-phenylethyl)ureate) bis(dimethylamido)titanium (Ti3). 50% probability, hydrogen atoms omitted for clarity.

FIG. 2 is a plot of conversion vs time for the reaction of N-methylaniline and 1-octene for catalysts generated in situ from ligands N-(2,6-diisopropylphenyl)benzamide (L5) (diamonds) and 3-(2,6-diisopropylphenyl)-1-methyl-1-(1-phenylethyl)urea (L7) (squares) and catalyst Ti3 (triangles).

FIG. 3 shows a photographs of a modified preparation outside of glovebox; top image: from left to right: 1-octene+N-methylaniline, Ti(NMe₂)₄, L7; middle left image: after addition of 1-octene and N-methylaniline to L7; middle right image: after addition of Ti(NMe₂)₄; bottom left image: after attachment of the balloon; and bottom right image: during the reaction.

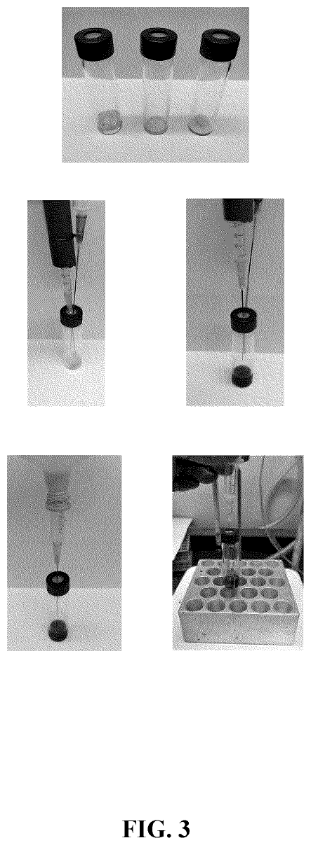

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

The term “suitable” as used herein means that the selection of the particular compound and/or conditions would depend on the specific synthetic manipulation to be performed, and/or the identity of the compound(s) to be transformed, but the selection would be well within the skill of a person skilled in the art. All method steps described herein are to be conducted under conditions sufficient to provide the product shown. A person skilled in the art would understand that all reaction conditions, including, for example, reaction solvent or lack thereof, reaction time, reaction temperature, reaction pressure, reactant ratio and whether or not the reaction should be performed under an anhydrous or inert atmosphere, can be varied to optimize the yield of the desired product and it is within their skill to do so.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

Terms of degree such as “substantially”, “about” and “approximately” as used herein mean a reasonable amount of deviation of the modified term such that the end result is not significantly changed. These terms of degree should be construed as including a deviation of at least +5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The abbreviation “Ad” as used herein refers to adamantyl.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₆alkyl means an alkyl group having 1, 2, 3, 4, 5 or 6 carbon atoms.

The term “alkylene” as used herein, whether it is used alone or as part of another group, means a straight or branched chain, bivalent form of an alkane, that is, a saturated carbon chain that links two other groups. The number of carbon atoms that are possible in the referenced alkylene group are indicated by the numerical prefix “Cₙ₁₋ₙ₂”. For example, the term C₁₋₆alkylene means an alkylene group having 1, 2, 3, 4, 5 or 6 carbon atoms.

The term “aryl” as used herein, whether it is used alone or as part of another group, refers to groups that contain at least one aromatic ring. When an aryl group contains more than one aromatic ring the term “aryl” as used herein includes condensed aromatic systems and moieties in which the aromatic rings are linked by a single bond. In an embodiment, the aryl group contains from 6, 9, 10 or 14 atoms, such as phenyl, naphthyl, indanyl or anthracenyl.

The term “arylene” as used herein, whether it is used alone or as part of another group, means a bivalent form of an aryl, that is, an aryl that links two other groups.

The term “cycloalkyl” as used herein, whether it is used alone or as part of another group, means a mono- or bicyclic, saturated cycloalkyl group. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. When a cycloalkyl group contains more than one cyclic structure or rings, the cyclic structures may be fused, bridged, spiro connected or linked by a single bond. The term “fused” as used herein in reference to a first cyclic structure being “fused” with a second cyclic structure means the first cyclic structure and the second cyclic structure share at least two adjacent atoms therebetween. The term “bridged” as used herein in reference to a first cyclic structure being “bridged” with a second cyclic structure means the first cyclic structure and the second cyclic structure share at least two non-adjacent atoms therebetween. The term “spiro-connected” in reference to a first cyclic structure being “spiro connected” with a second cyclic structure means the first cyclic structure and the second cyclic structure share one atom therebetween.

The term “cycloalkylene” as used herein, whether it is used alone or as part of another group, means a bivalent form of a cycloalkane, that is, a saturated cycloalkane that links two other groups. The number of carbon atoms that are possible in the referenced cycloalkylene group are indicated by the numerical prefix “Cₙ₁₋ₙ₂”. For example, the term C₃₋₄₀cycloalkylene means a cycloalkylene group having 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 carbon atoms.

The abbreviation “Dipp” as used herein refers to 2,6-diisopropylphenyl.

The term “halo” as used herein refers to a halogen atom and includes F, Cl and Br.

The term “haloalkyl” as used herein refers to an alkyl group wherein one or more, including all of the available hydrogen atoms are replaced by a halogen atom. The number of carbon atoms that are possible in the referenced haloalkyl group are indicated by the numerical prefix “Cₙ₁₋ₙ₂”. For example, the term C₁₋₆haloalkyl means a haloalkyl group having 1, 2, 3, 4, 5 or 6 carbon atoms. In an embodiment, the halogen is a fluorine, in which case the haloalkyl is optionally referred to herein as a “fluoroalkyl” group. In an embodiment of the present disclosure, all of the hydrogen atoms are replaced by fluorine atoms.

The term “heterocycle” as used herein, whether it is used alone or as part of another group, refers to a ring-containing group having one or more multivalent heteroatoms (for example, nitrogen), as a part of the ring structure and including at least 3 and up to 20 atoms in the ring(s).

The term “substituted” as used herein refers to a moiety wherein one or more, including all of the available hydrogens are replaced by a non-hydrogen group.

### II. Methods for Hydroaminoalkylation

An earth-abundant titanium catalyst can be readily synthesized, for example by using commercially available and inexpensive Ti(NMe₂)₄ and a ureate or amidate ligand in situ. For example, the system using a sterically demanding ureate ligand exhibited exceptional turnover frequencies (TOFs) in comparison to reported titanium catalysts based upon Ti(NMe₂)₄ for a wide range of hydroaminoalkylation substrates and can even complete reactions with challenging substrates such as dialkylamines within a day. Furthermore, such catalysts may allow, for example, for new handling methods for hydroaminoalkylation without the necessity of glovebox techniques and a wide-ranging practical applicability in the “green” synthesis, for example, of amine-functionalized materials, amino acids and N-heterocyclic building blocks for medicinal chemistry.

Accordingly, the present disclosure includes a method for the hydroaminoalkylation of an olefin, the method comprising reacting the olefin with a secondary amine in the presence of a catalyst of Formula (I):

LₓM(R¹)_{y}(I),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L is a ligand of Formula (II)

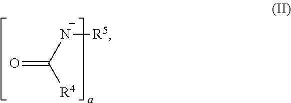

wherein
each a is independently 1 or 2;
each R⁴ is independently C₁₋₄₀alkyl, C₃₋₄₀cycloalkyl, substituted or unsubstituted aryl
or NR⁶R⁷;
   (i) when a is 1,
      R⁵ is C₁₋₄₀alkyl, C₃₋₄₀cycloalkyl or substituted or unsubstituted aryl; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and
      R⁶ is C₁₋₄₀alkyl, C₃₋₄₀cycloalkyl, substituted or unsubstituted aryl or C₁₋₁₀alkylene-aryl, wherein the aryl is substituted or unsubstituted;
   (ii) when a is 2,
      R⁵ is C₁₋₄₀alkylene, C₃₋₄₀cycloalkylene or substituted or unsubstituted arylene; and R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

In an embodiment, a is 1. In another embodiment, a is 2. In an embodiment, x is 1 to 3. In another embodiment, x is 1 or 2. In an embodiment, x is 1. In another embodiment, x is 2. It will be appreciated by a person skilled in the art that a and x are any suitable combination. It will also be appreciated by a person skilled in the art that while the ligand of Formula (II) is depicted herein with a negative charge on the nitrogen, amidate and ureate ligands can adopt a number of modes of coordination to the group 4 metal M therefore the present disclosure includes all possible modes of coordination including monodentate (i.e. through a nitrogen or oxygen donor) and bidentate (e.g. through both a nitrogen and an oxygen donor) binding motifs.

In an embodiment, M is Ti (titanium), Zr (zirconium) or Hf (hafnium). In another embodiment, M is Ti or Zr. In a further embodiment, M is Ti.

In an embodiment, R¹ is N(R²)₂. In another embodiment, R¹ is —C₁₋₆alkylene-Si(R³)₃. In an embodiment, R² and R³ are each independently C₁₋₄alkyl. In another embodiment, R² and R³ are each methyl. Accordingly, in another embodiment, R¹ is NMe₂ or CH₂Si(CH₃)₃. In another embodiment, R¹ is NMe₂. In another embodiment, R¹ is CH₂Si(CH₃)₃.

In an embodiment R⁴ is NR⁶R⁷, and R⁶ and R⁷ are each independently C₁₋₁₆alkyl, C₃₋₁₆cycloalkyl, substituted or unsubstituted aryl or C₁₋₆alkylene-aryl. In another embodiment, R⁶ and R⁷ are each independently C₁₋₆alkyl, unsubstituted aryl or C₁₋₆alkylene-aryl. In another embodiment, R⁶ and R⁷ are different. In a further embodiment, R⁶ is C₁₋₆alkyl and R⁷ is C₁₋₄ alkylene-aryl. In another embodiment, R⁶ is methyl and R⁷ is —CH(CH₃)-phenyl.

In an alternative embodiment, R⁴ is C₁₋₄₀alkyl, C₃₋₄₀cycloalkyl or substituted or unsubstituted aryl. In another embodiment, R⁴ is C₁₋₁₆alkyl or substituted or unsubstituted aryl. In an embodiment, the substituted aryl is 4-substituted phenyl, 3,5-disubstituted phenyl or 2,4,6-trisubstituted phenyl. In an embodiment, the substituents on the aryl (e.g. phenyl) are independently selected from C₁₋₆alkyl and C₁₋₆fluoroalkyl. In another embodiment, the substituents on the aryl (e.g. phenyl) are independently selected from methyl and trifluoromethyl. In another embodiment, the substituents on the aryl (e.g. phenyl) are each methyl or are each trifluoromethyl. In an embodiment, R⁴ is methyl, t-butyl, adamantyl, phenyl, 4-trifluoromethylphenyl, 3,5-bis(trifluoromethyl)phenyl or 2,4,6-trimethylphenyl. In another embodiment, R⁴ is C₁₋₁₀alkyl or unsubstituted aryl. In another embodiment, R⁴ is tert-butyl or phenyl. In an embodiment, R⁴ is tert-butyl. In another embodiment of the present disclosure, R⁴ is phenyl.

In an embodiment, R⁵ is C₁₋₄₀alkyl, C₃₋₄₀cycloalkyl or substituted or unsubstituted aryl. In another embodiment, R⁵ is C₁₋₁₆alkyl, C₃₋₁₆cycloalkyl or substituted aryl. In an embodiment, the substituted aryl is 2,6-disubstituted phenyl or 3,5-disubstituted phenyl. In another embodiment, the substituted aryl is 2,6-disubstituted phenyl. In an embodiment, the substituents on the aryl (e.g. phenyl) are independently selected from C₁₋₆alkyl and C₁₋₆haloalkyl. In an embodiment, the substituents on the aryl (e.g. phenyl) are independently selected from C₁₋₆alkyl and C₁₋₆fluoroalkyl. In another embodiment, the substituents on the aryl (e.g. phenyl) are independently selected from methyl, trifluoromethyl and isopropyl. In another embodiment, the substituents on the aryl (e.g. phenyl) are each methyl, are each trifluoromethyl or are each isopropyl. In a further embodiment, the substituents on the aryl (e.g. phenyl) are each isopropyl. In an embodiment, R⁵ is t-butyl, adamantyl, 2,6-diisopropylphenyl, 2,6-dimethylphenyl or 3,5-bis(trifluoromethyl)phenyl. In an embodiment, R⁵ is substituted aryl. In another embodiment, R⁵ is 2,6-diisopropylphenyl.

In an embodiment, R⁵ and R⁷ are bonded together, thereby forming, together with the nitrogen atom they are both bound to, a substituted or unsubstituted heterocycle. In an embodiment, the heterocycle is unsubstituted. In another embodiment, the heterocycle is a 5-membered ring. In such embodiments, it is an embodiment that R⁶ is C₁₋₁₆alkyl or unsubstituted aryl. In another embodiment of the present disclosure, R⁶ is t-butyl or phenyl.

In an embodiment, R⁵ is C₁₋₄₀alkylene, C₃₋₄₀cycloalkylene or substituted or unsubstituted arylene. In another embodiment, R⁵ is C₁₋₄₀alkylene or unsubstituted arylene. In another embodiment, R⁵ is C₁₋₁₆alkylene or unsubstituted arylene. In another embodiment of the present disclosure, R⁵ is C₁₋₆alkylene or unsubstituted arylene. In another embodiment, R⁵ is —CH₂C(CH₃)₂CH₂— or

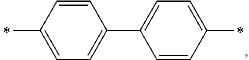

wherein * indicates the sites of attachment of R⁵ to the remainder of the ligand of Formula (II). In another embodiment, R⁵ is —CH₂C(CH₃)₂CH₂—. In a further embodiment, R⁵ is

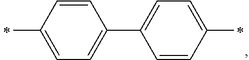

wherein * indicates the sites of attachment of R⁵ to the remainder of the ligand of Formula (II).

The catalyst of Formula (I) can be prepared by any suitable means. In an embodiment, the catalyst is prepared from the reaction of a compound of Formula (III) with a group 4 metal complex of Formula (IV):

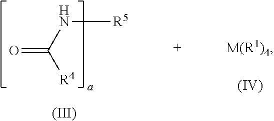

wherein M, R¹, R⁴, R⁵ and a are as defined in the present disclosure.

However, alternative methods of preparing the catalyst of Formula (I) can also be employed. For example, in an embodiment, the catalyst is prepared from a method comprising salt metathesis of a group 4 metal halide (e.g. TiCl₄) with a suitable salt of a ligand of Formula (II) as defined herein followed by a ligand substitution reaction with a reagent of the formula A-R¹, wherein A is of the formula —Mg—X (wherein X is halo such as Cl or Br) or Li and R¹ is as defined herein. In another embodiment, the catalyst is prepared from a method comprising salt metathesis of a group 4 metal compound of the formula TiX₂(R¹)₂ (e.g. wherein X is halo such as Cl) and R¹ is as defined herein with a suitable salt of a ligand of Formula (II) as defined herein.

In an embodiment, the compound of Formula (III) is selected from:

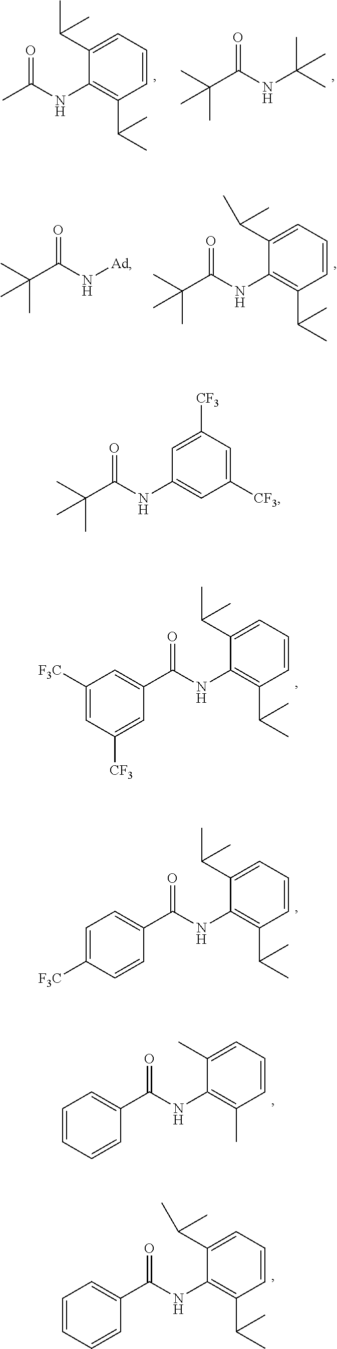

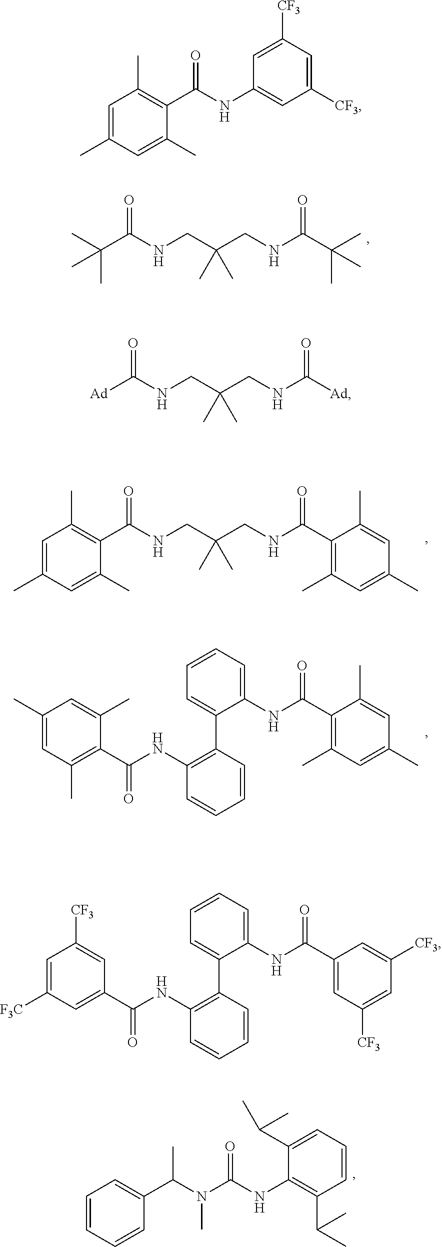

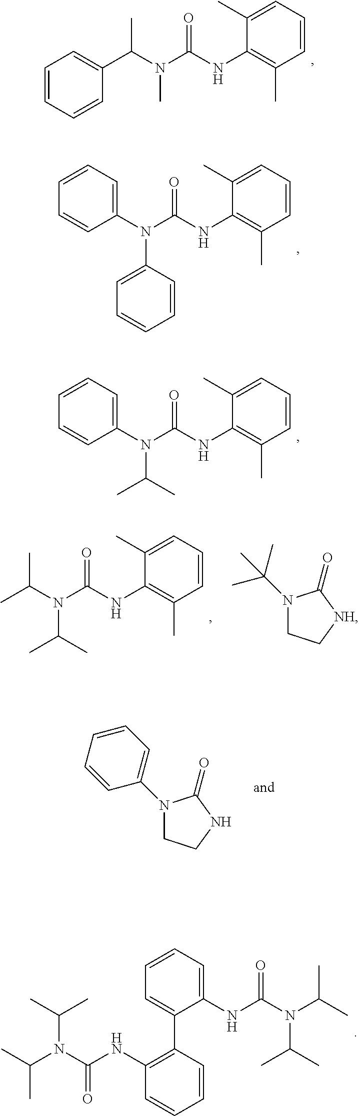

In another embodiment, the compound of Formula (III) is selected from:

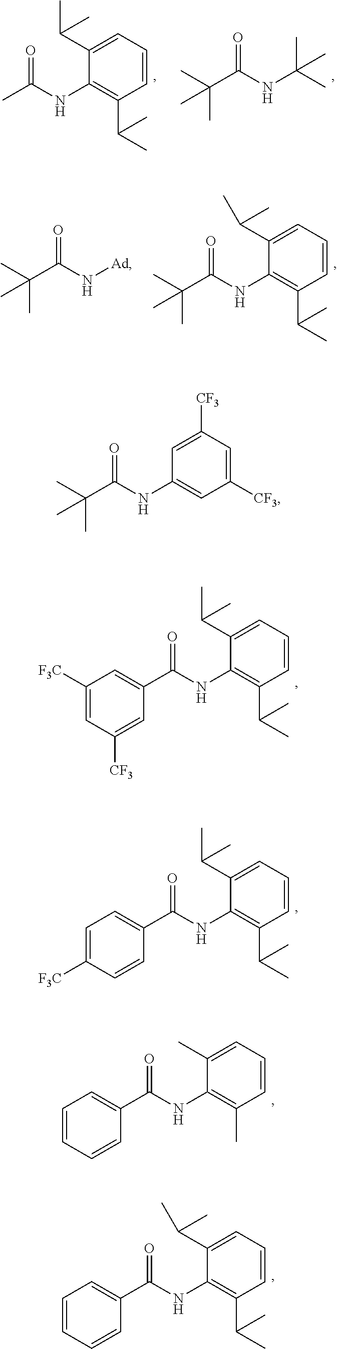

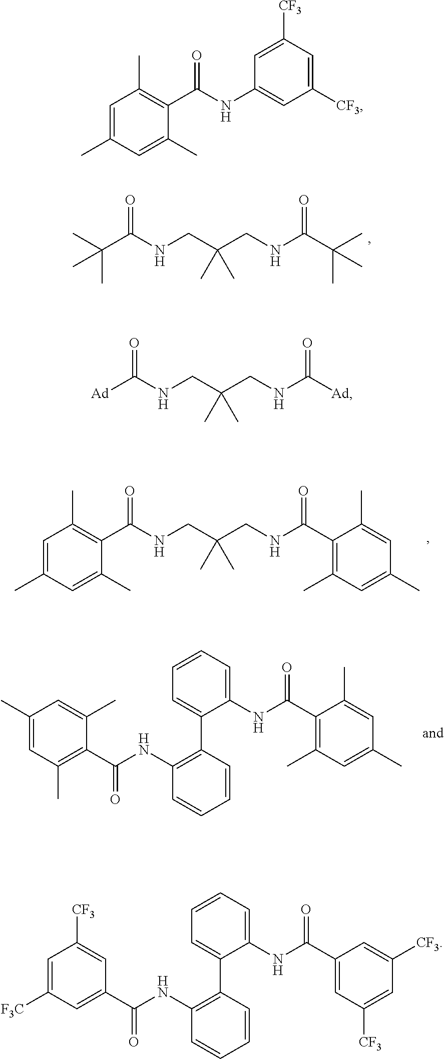

In another embodiment, the compound of Formula (III) is selected from:

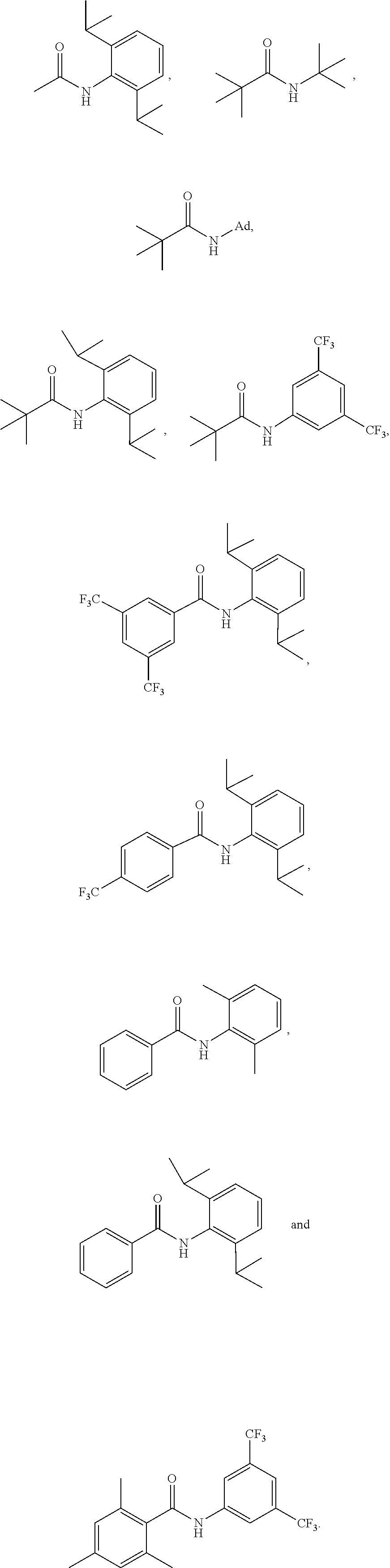

In another embodiment, the compound of Formula (III) is selected from:

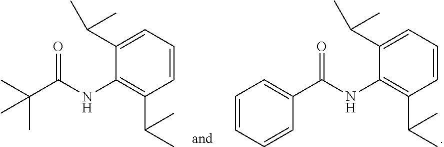 [SMILES: CC(C)c1cccc(C(C)C)c1NC(=O)C(C)(C)C ; CC(C)c1cccc(C(C)C)c1NC(=O)c1ccccc1]

In another embodiment, the compound of Formula (III) is:

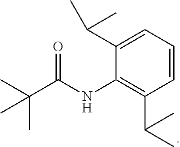 [SMILES: CC(C)c1cccc(C(C)C)c1NC(=O)C(C)(C)C]

In another embodiment, the compound of Formula (III) is:

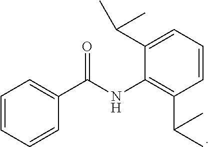 [SMILES: CC(C)c1cccc(C(C)C)c1NC(=O)c1ccccc1]

In another embodiment, the compound of Formula (III) is selected from:

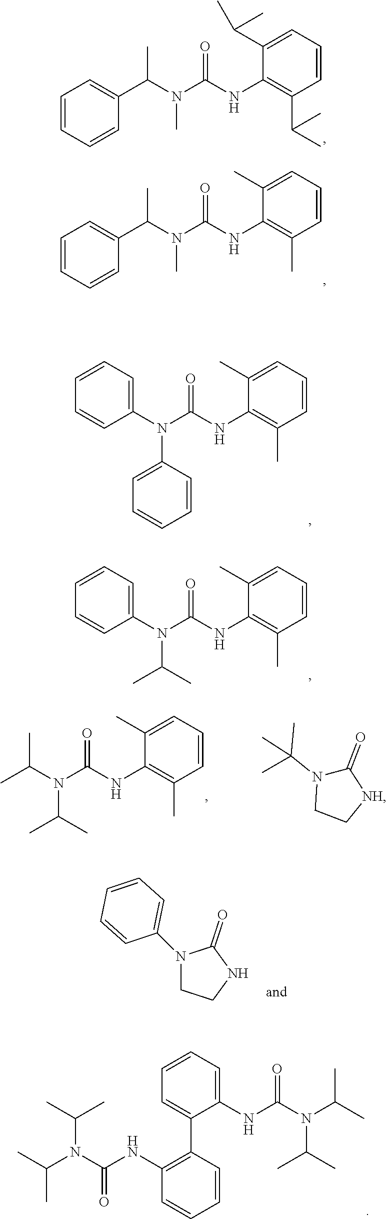

In another embodiment, the compound of Formula (III) is selected from:

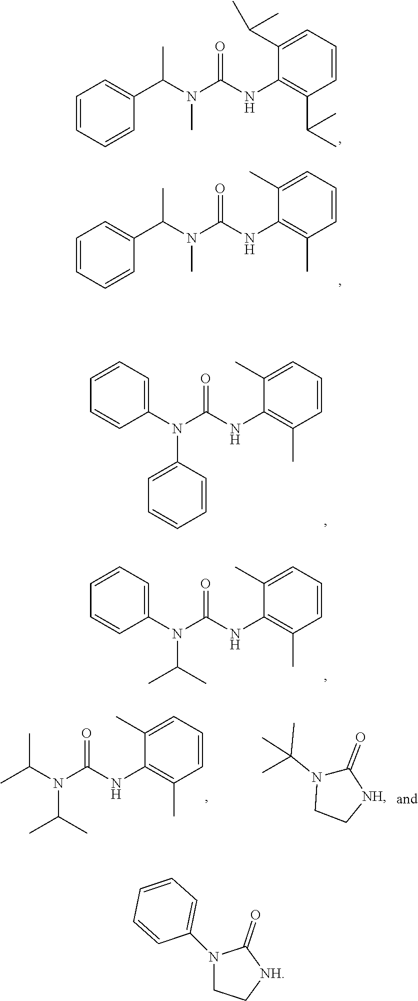

In another embodiment, the compound of Formula (III) is selected from:

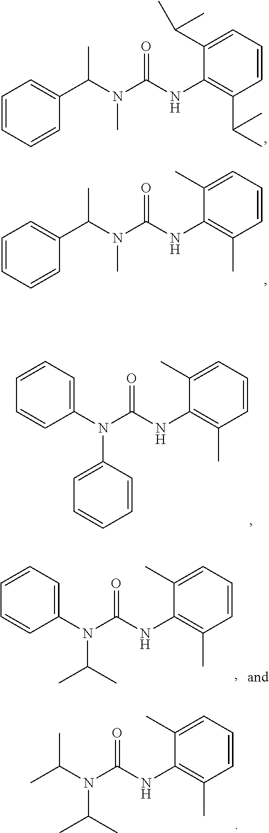

In another embodiment, the compound of Formula (III) is selected from:

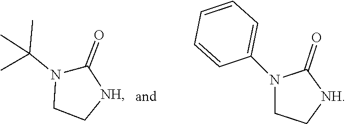

In another embodiment, the compound of Formula (III) is selected from:

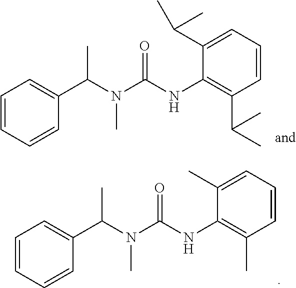

In another embodiment, the compound of Formula (III) is:

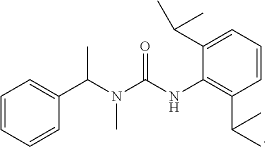 [SMILES: CC(C)c1cccc(C(C)C)c1NC(=O)N(C)C(C)c1ccccc1]

In another embodiment, the compound of Formula (III) is:

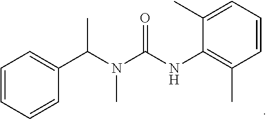 [SMILES: Cc1cccc(C)c1NC(=O)N(C)C(C)c1ccccc1]

It will be appreciated by a person skilled in the art that embodiments of the ligand of Formula (II) can be varied as described herein for the compound of Formula (III) but are the corresponding deprotonated form of the chemical structures shown hereinabove.

In an embodiment, the catalyst is generated in situ. In another embodiment, the method comprises use of a pre-prepared catalyst of Formula (I).

In an embodiment, the reaction of the olefin with the secondary amine is carried out without addition of solvent. The term “without addition of solvent” as used herein includes reactions with trace or small amounts of solvent, for example where a minimal or small volume of solvent is used to dissolve the catalyst. In an alternative embodiment, the reaction of the olefin with the secondary amine is carried out in a suitable solvent or mixtures thereof such as toluene.

The reaction of the olefin with the secondary amine is carried out at a temperature, for a time and using a catalyst loading suitable for the hydroaminoalkylation proceed to a sufficient extent. In an embodiment, the reaction of the olefin with the secondary amine is carried out a temperature of from about 140° C. to about 180° C. or about 160° C. It will be appreciated by a person skilled in the art, for example, with reference to the Examples herein that a suitable time and/or catalyst loading may depend, for example, on the identity of the olefin and/or the secondary amine and the skilled person would be able to select a suitable time and/or a suitable method for the determination whether the reaction had proceeded to a sufficient extent as well as a suitable catalyst loading. In an embodiment, the reaction of the olefin with the secondary amine is carried out for a time of from about 1 hour to about 96 hours or about 1 hour to about 72 hours or less than about 8 hours. In another embodiment, the catalyst loading is from about 1 mol % to about 30 mol %, about 5 mol % to about 20 mol %, about 5 mol % to about 10 mol %, about 5 mol % or about 10 mol %.

The olefin can be any suitable olefin including olefins that are hydrocarbons as well as those having one or more functional groups that are desirably stable under the conditions used for the hydroaminoalkylation or are alternatively protected by a suitable protecting group. The term “protecting” as used herein refers to using a chemical moiety, that is a “protecting group” which protects or masks a reactive portion of a molecule to prevent side reactions in that reactive portion of the molecule, while manipulating or reacting a different portion of the molecule. After the manipulation or reaction is complete, the protecting group is removed under conditions that do not degrade or decompose the remaining portions of the molecule; i.e. the protected reactive portion of the molecule is “deprotected”. The selection of a suitable protecting group can be made by a person skilled in the art. Many conventional protecting groups are known in the art, for example as described in “Protective Groups in Organic Chemistry” McOmie, J. Ed., Plenum Press, 1973, in Greene, T. W. and Wuts, P. G. M., “Protective Groups in Organic Synthesis”, John Wiley & Sons, 4th Edition, 2006 and in Kocienski, P. “Protecting Groups”, 3rd Edition, 2003, Georg Thieme Verlag (The Americas). In an embodiment, the olefin comprises a terminal alkene. In another embodiment, the olefin comprises an internal alkene. In an embodiment, the olefin further comprises a silyl protected alcohol. In another embodiment, the olefin comprises a cyclic alkene; i.e. the olefin comprises one or more sites of unsaturation in a non-aromatic ring. Certain cyclic alkenes such as substituted and unsubstituted cyclooctadienes and norbomadienes can be used as the olefin in the methods of the present disclosure to prepare amine-substituted monomers that can be used in ring opening metathesis polymerization (ROMP) to prepare amine-functionalized polymers. For example, a person skilled in the art would readily appreciate that suitable monomers for ROMP comprise a cyclic olefin wherein the driving force of the reaction is relief of ring strain of the cyclic olefin. Suitable conditions for the ROMP of amine-substituted monomers is described for example, in PCT Application Publication No. 2019/222852 filed on 23 May 2019. Accordingly, in an embodiment, the reaction of the olefin with the secondary amine produces an amine-substituted monomer for ring-opening metathesis polymerization (ROMP). In another embodiment, the olefin is a substituted or unsubstituted cyclooctadine or a substituted or unsubstituted norbomadiene. In another embodiment, the olefin is an unsubstituted cyclooctadine or an unsubstituted norbomadiene. In a further embodiment, the olefin is an unsubstituted cyclooctadiene. In another embodiment, the olefin is an unsubstituted norbomadiene. Alternatively, amine-substituted polymers can be prepared by the hydroaminoalkylation of suitable polymers comprising an alkene. Such polymers can comprise, for example, pendant alkene moieties or terminal alkene moieties. In another embodiment, the polymer is an alkene-terminated polyolefin. In an embodiment, the polyolefin is a polyethylene or a polypropylene. In another embodiment, the polyolefin is an atactic polypropylene. In an embodiment, the number average molecular weight of the alkene-terminated polyolefin is about 300 g/mol to about 10,000 g/mol, about 350 g/mol to about 8,500 g/mol, about 350 g/mol, about 850 g/mol or about 8,500 g/mol.

In another embodiment, the olefin is selected from: norbomene,

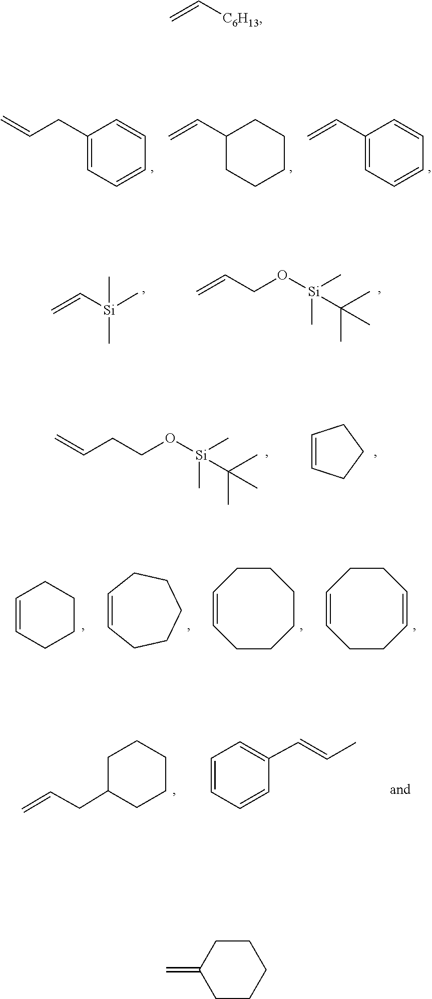

or any combination thereof.

The secondary amine can be any suitable secondary amine. For example, it will be appreciated by a person skilled in the art that a suitable secondary amine for a hydroaminoalkylation comprises a C—H bond α- to its nitrogen. In an embodiment, the secondary amine is of the formula NR^{a}R^{b}, wherein R^{a} and R^{b} are each independently substituted or unsubstituted C₁₋₄₀alkyl, C₃₋₄₀cycloalkyl, C₁₋₁₀alkylene-C₃₋₄₀cycloalkyl, aryl or C₁₋₁₀alkylene-aryl or R^{a} and R^{b} are bonded together, thereby forming, together with the nitrogen atom they are both bound to, a substituted or unsubstituted heterocycle that is optionally part of a condensed ring system. In another embodiment, R^{a} is C₁₋₄alkyl and R^{b} is substituted or unsubstituted aryl. In a further embodiment, the amine is of the formula

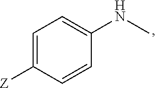

wherein Z is H, halo, —O—C₁₋₁₀alkyl, —O—C₃₋₁₀cycloalkyl, C₁₋₁₀alkyl or C₃₋₁₀cycloalkyl. In another embodiment, Z is H, Cl, Br or —OMe. In another embodiment, the secondary amine is selected from:

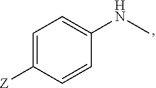

wherein Z is H, —OCH₃, Cl or Br;

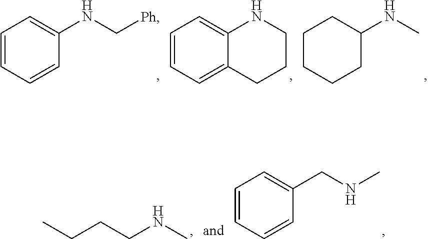

or any combination thereof.

In another embodiment, the hydroaminoalkylation is intramolecular; i.e. the amine and the olefin are moieties that are present in the same compound.

The present disclosure also includes a method for the preparation of a catalyst of Formula (I), the method comprising reaction of a compound of Formula (III) with a group 4 metal complex of Formula (IV):

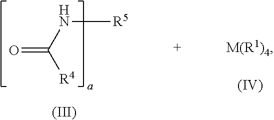

wherein M, R¹, R⁴, R⁵ and a are as defined in the present disclosure. In an embodiment, the catalyst of Formula (I) is a compound of Formula I(a) as defined herein.

The present disclosure also includes a method for the hydroaminoalkylation of an olefin, the method comprising reacting the olefin with a secondary amine in the presence of a compound of Formula I(a), wherein the compound of Formula I(a) is as defined herein.

The present disclosure also includes a hydroaminoalkylated olefin prepared by a method for the hydroaminoalkylation of the olefin as described herein.

### III. Compounds

The present disclosure also includes a compound of Formula I(a):

(L^{a})ₓM(R¹)_{y}I(a),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L^{a} is a ligand of Formula II(a)

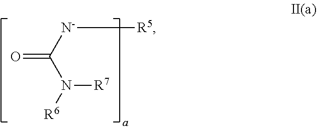

wherein
each a is independently 1 or 2;
   (i) when a is 1,
      R⁵ is C₁₋₄₀alkyl, C₃₋₄₀cycloalkyl or substituted or unsubstituted aryl; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and R⁶ is C₁₋₄₀alkyl, C₃₋₄₀cycloalkyl, substituted or unsubstituted aryl or C₁₋₁₀alkylene-aryl, wherein the aryl is substituted or unsubstituted;
   (ii) when a is 2,
      R⁵ is C₁₋₄₀alkylene, C₃₋₄₀cycloalkylene or substituted or unsubstituted arylene; and
      R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

In an embodiment, a is 1. In another embodiment, a is 2. In an embodiment, x is 1 to 3. In another embodiment, x is 1 or 2. In an embodiment, x is 1. In another embodiment, x is 2. It will be appreciated by a person skilled in the art that a and x are any suitable combination. It will also be appreciated by a person skilled in the art that while the ligand of Formula II (a) is depicted herein with a negative charge on the nitrogen, ureate ligands can adopt a number of modes of coordination to the group 4 metal M therefore the present disclosure includes all possible modes of coordination including monodentate (i.e. through a nitrogen or oxygen donor) and bidentate (e.g. through both a nitrogen and an oxygen donor) binding motifs.

In an embodiment, M is Ti (titanium), Zr (zirconium) or Hf (hafnium). In another embodiment, M is Ti or Zr. In a further embodiment, M is Ti.

In an embodiment, R¹ is N(R²)₂. In another embodiment, R¹ is —C₁₋₆alkylene-Si(R³)₃. In an embodiment, R² and R³ are each independently C₁₋₄alkyl. In another embodiment, R² and R³ are each methyl. Accordingly, in another embodiment, R¹ is NMe₂ or CH₂Si(CH₃)₃. In another embodiment, R¹ is NMe₂. In another embodiment, R¹ is CH₂Si(CH₃)₃.

In an embodiment, R⁶ and R⁷ are each independently C₁₋₁₆alkyl, C₃₋₁₆cycloalkyl, substituted or unsubstituted aryl or C₁₋₆alkylene-aryl. In another embodiment, R⁶ and R⁷ are each independently C₁₋₆alkyl, unsubstituted aryl or C₁₋₆alkylene-aryl. In another embodiment, R⁶ and R⁷ are different. In a further embodiment, R⁶ is C₁₋₆alkyl and R⁷ is C₁₋₄alkylene-aryl. In another embodiment, R⁶ is methyl and R⁷ is —CH(CH₃)-phenyl.

In an embodiment, R⁵ is C₁₋₄₀alkyl, C₃₋₄₀cycloalkyl or substituted or unsubstituted aryl. In another embodiment, R⁵ is C₁₋₁₆alkyl, C₃₋₁₆cycloalkyl or substituted aryl. In an embodiment, the substituted aryl is 2,6-disubstituted phenyl or 3,5-disubstituted phenyl. In another embodiment, the substituted aryl is 2,6-disubstituted phenyl. In an embodiment, the substituents on the aryl (e.g. phenyl) are independently selected from C₁₋₆alkyl and C₁₋₆haloalkyl. In an embodiment, the substituents on the aryl (e.g. phenyl) are independently selected from C₁₋₆alkyl and C₁₋₆fluoroalkyl. In another embodiment, the substituents on the aryl (e.g. phenyl) are independently selected from methyl, trifluoromethyl and isopropyl. In another embodiment, the substituents on the aryl (e.g. phenyl) are each methyl, are each trinfluoromethyl or are each isopropyl. In a further embodiment, the substituents on the aryl (e.g. phenyl) are each isopropyl. In an embodiment, R⁵ is t-butyl, adamantyl, 2,6-diisopropylphenyl, 2,6-dimethylphenyl or 3,5-bis(trifluoromethyl)phenyl. In another embodiment, R⁵ is 2,6-diisopropylphenyl or 2,6-dimethylphenyl. In an embodiment, R⁵ is substituted aryl. In another embodiment, R⁵ is 2,6-diisopropylphenyl.

In an embodiment, R⁵ and R⁷ are bonded together, thereby forming, together with the nitrogen atom they are both bound to, a substituted or unsubstituted heterocycle. In an embodiment, the heterocycle is unsubstituted. In another embodiment, the heterocycle is a 5-membered ring. In such embodiments, it is an embodiment that R⁶ is C₁₋₁₆alkyl or unsubstituted aryl. In another embodiment of the present disclosure, R⁶ is t-butyl or phenyl.

In an embodiment, R⁵ is C₁₋₄₀alkylene, C₃₋₄₀cycloalkylene or substituted or unsubstituted arylene. In another embodiment, R⁵ is C₁₋₄₀alkylene or unsubstituted arylene. In another embodiment, R⁵ is C₁₋₁₆alkylene or unsubstituted arylene. In another embodiment of the present disclosure, R⁵ is C₁₋₆alkylene or unsubstituted arylene. In another embodiment, R⁵ is
—CH₂C(CH₃)₂CH₂— or

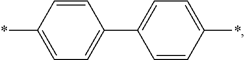

wherein * indicates the sites of attachment of R⁵ to the remainder of the ligand of Formula II(a). In another embodiment, R⁵ is —CH₂C(CH₃)₂CH₂—. In a further embodiment, R⁵ is

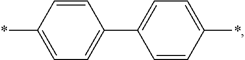

wherein * indicates the sites of attachment of R⁵ to the remainder of the ligand of Formula II(a).

In an embodiment, the ligand of Formula II(a) is selected from the corresponding deprotonated forms of the following compounds of Formula (III):

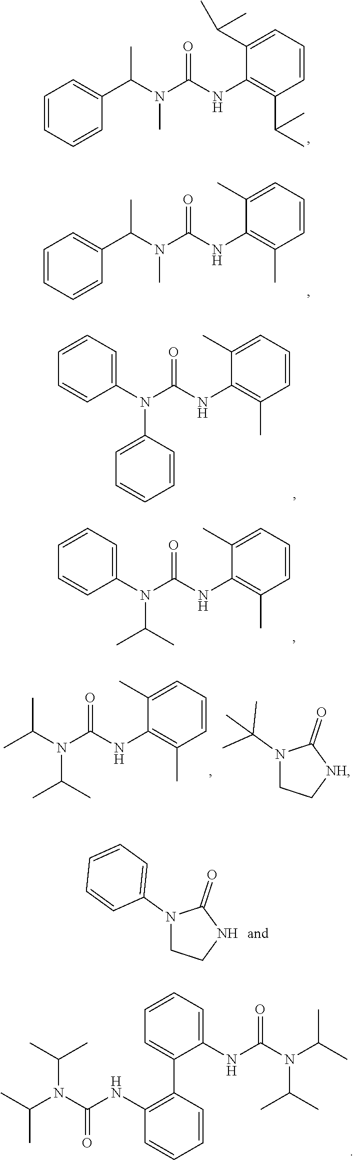

In another embodiment, the ligand of Formula II(a) is selected from the corresponding deprotonated forms of the following compounds of Formula (III):

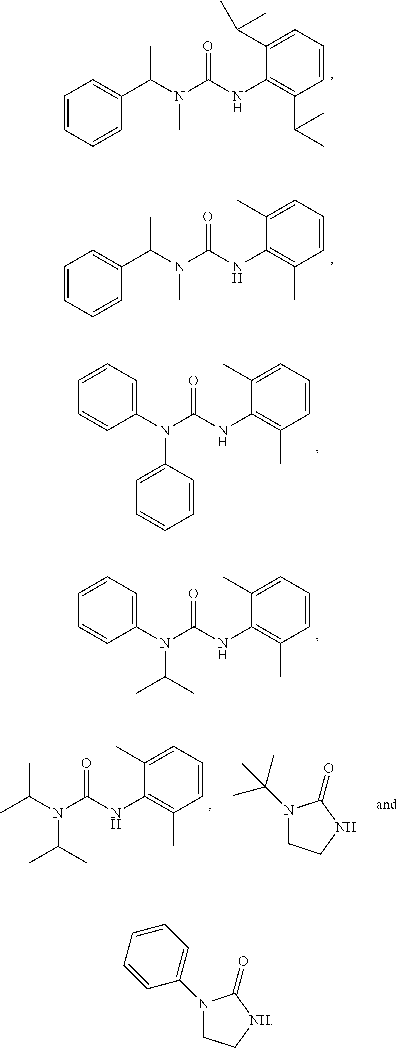

In another embodiment, the ligand of Formula II(a) is selected from the corresponding deprotonated forms of the following compounds of Formula (III):

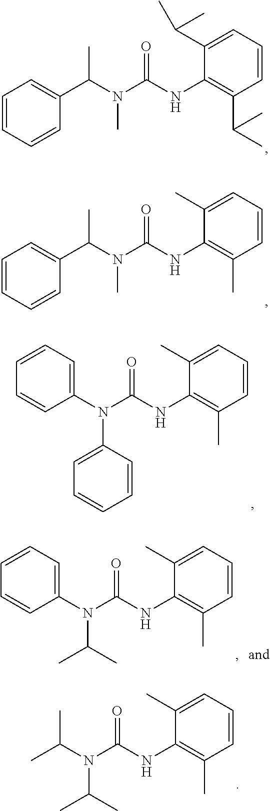

In another embodiment, the ligand of Formula II(a) is selected from the corresponding deprotonated forms of the following compounds of Formula (III):

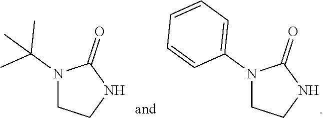

In another embodiment, the ligand of Formula II(a) is selected from the corresponding deprotonated forms of the following compounds of Formula (III):

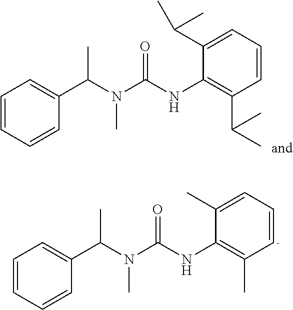 [SMILES: CC(C)c1cccc(C(C)C)c1NC(=O)N(C)C(C)c1ccccc1 ; Cc1cccc(C)c1NC(=O)N(C)C(C)c1ccccc1]

In another embodiment, the ligand of Formula II(a) is the corresponding deprotonated form of the following compound of Formula (III):

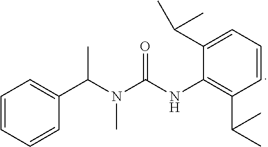 [SMILES: CC(C)c1cccc(C(C)C)c1NC(=O)N(C)C(C)c1ccccc1]

In another embodiment, the ligand of Formula II(a) is the corresponding deprotonated form of the following compound of Formula (III):

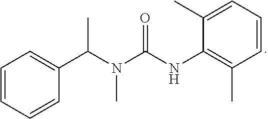 [SMILES: Cc1cccc(C)c1NC(=O)N(C)C(C)c1ccccc1]

The following non-limiting examples are illustrative of the present disclosure:

### EXAMPLES

### Example 1: Synthesis and Reactivity of Titanium Catalysts for Hydroaminoalkylation

An object was to develop a titanium catalyst for hydroaminoalkylation, which not only preserves most of reactivity of the highest state of the art systems, but desirably improves the user-friendliness significantly with a catalyst which may, for example, be generated in situ with use of easily synthesized ligands and readily available titanium sources. A broad range of 1,3-N,X-chelated catalysts was screened to identify ligand classes, which provided the most active systems. A difference in reactivity of isolated versus in situ prepared catalysts was observed with this set of hemilabile ligands. A high reactivity and selectivity for the branched hydroaminoalkylation product through various transformations of unactivated terminal alkenes with alkyl and aryl amines was demonstrated. Finally, a variety of reaction procedures using benchtop techniques is described in regard to their applications, for example, in the field of monomer design for N-containing polymers and/or synthesis of building blocks for medicinally relevant small molecules.

### A. Materials and Methods

### I. General Information

All reactions were performed under a N₂ atmosphere using Schlenk or glovebox techniques, unless otherwise stated. Chemicals without a detailed synthesis description in the Synthesis and Characterization of Compounds section below were purchased from commercial sources and used without further purification, unless otherwise noted. All amines and alkenes were dried over CaH₂ and distilled and degassed prior to use in catalytic experiments. Solvents were dried according to standard procedures and stored over activated molecular sieves (4 A). Toluene-d₈ was dried over sodium-benzophenone ketyl and distilled prior to use. Experiments conducted on an NMR tube scale were performed in J. Young NMR tubes (8″×5 mm) sealed with screw-type Teflon™ caps. All glassware was dried in a 180° C. oven overnight before use.

¹H and ¹³C NMR spectra were recorded on Bruker 300 MHz and 400 MHz Avance™ spectrometers at ambient temperature. Chemical shifts (( ) are given relative to residual protons of the solvent and are reported in parts per million (ppm). Coupling constants J are given in Hertz (Hz). The following abbreviations are used to indicate signal multiplicity: s=singlet, d=doublet, t=triplet, q=quartet, m=multiplet, and br=broad. High resolution mass-spectra (HRMS) were measured by the mass spectrometry services at University of British Columbia on a Kratos MS-50 spectrometer using a Bruker maXis™ ultra-high resolution tandem time-of-flight (UHR-Qq-TOF) mass spectrometer using a positive electrospray ionization source. Fragment signals are given in mass per charge number (m/z). Gas chromatography-mass spectrometry (GC-MS) analyses were conducted on an Agilent 7890B GC with an Agilent 5977 inert CI mass detector, utilizing methane as the ionization gas. Single-crystal X-ray structure determination was performed on an APEX II diffractometer at the Department of Chemistry, University of British Columbia.

### II. Synthesis and Characterization of Compounds

### (a) Ligands

### (i) N-tert-butoxycarbonyl-2,6-dimethylaniline (L2)

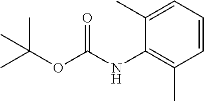 [SMILES: Cc1cccc(C)c1NC(=O)OC(C)(C)C]

L2 was prepared following a literature procedure.²¹ The chemical shifts for the title compound matched those previously reported.

### (ii) N-(2,6-diisopropylphenyl)-2-methylpropane-2-sulfinamide (L3)

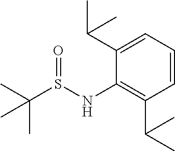 [SMILES: CC(C)c1cccc(C(C)C)c1NS(=O)C(C)(C)C]

L3 was prepared following a modified literature procedure.²² In particular, tert-butylsulfinyl chloride (500 mg, 3.56 mmol) in dichloromethane (3 M) was added dropwise to a solution of 2,6-diisopropylaniline (1.26 g, 7.11 mmol) in dichloromethane (1M) at −78° C. The mixture was stirred for 3 hours at room temperature (rt). The solution was filtered, and the filtrate was washed three times with water then dried over MgSO₄. The solution was concentrated by rotary evaporation and the crude product was purified by chromatography (SiO₂, n-hexane:ethyl acetate (EA)=1:1) and L3 was isolated as a colorless solid (620 mg, 2.20 mmol, 62%). ¹H NMR (CDCl₃, 400 MHz, rt): δ=1.23-1.27 (m, 12H, CH—(CH₃)₂), 1.44 (s, 9H, C—(CH₃)₃), 3.44 (h, J=6.8 Hz, 2 H, CH—(CH₃)₂), 5.31 (s, 1H, NH), 7.15-7.24 (m, 3H, Ar—H) ppm. ¹³C NMR (CDCl₃, 101 MHz, rt): δ=22.86 (CH—(CH₃)₂), 23.73, 23.90 (CH—(CH₃)₂), 27.93 (C—(CH₃)₃), 56.93 (C—(CH₃)₃), 123.86 (Ar—CHₘ), 126.73 (Ar—CHₚ), 134.23 (Ar—Cᵢ), 144.64 (Ar—Co) ppm. HRMS (ESI): m/z calc. for C₂₂H₃₁N₂O [M+H⁺]: 339.2437. Found: 339.2444. EA: calc. for C₁₆H₂₇NOS: C, 68.28; H, 9.67; N, 4.98; S, 11.39. Found: C, 68.60; H, 9.67; N, 4.99; S, 11.35.

### (iii) N-(2,6-diisopropylphenyl)pivalamide (L4)

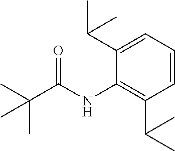 [SMILES: CC(C)c1cccc(C(C)C)c1NC(=O)C(C)(C)C]

L4 was prepared following a literature procedure.²³ The chemical shifts for the title compound matched those previously reported.

### (iv) N-(2,6-diisopropylphenyl)benzamide (L5)

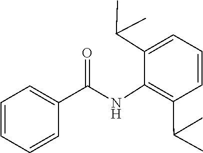 [SMILES: CC(C)c1cccc(C(C)C)c1NC(=O)c1ccccc1]

L5 was prepared following a literature procedure.²³ The chemical shifts for the title compound matched those previously reported.

### (v) 3-(2,6-dimethylphenyl)-1-methyl-1-(1-phenylethyl)urea (L6)

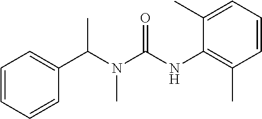 [SMILES: Cc1cccc(C)c1NC(=O)N(C)C(C)c1ccccc1]

L6 was prepared following a literature procedure.²⁴ The chemical shifts for the title compound matched those previously reported.

### (vi) 3-(2,6-diisopropylphenyl)-1-methyl-1-(1-phenylethyl)urea (L7)

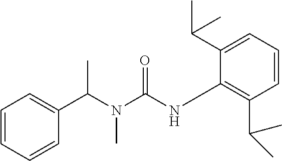 [SMILES: CC(C)c1cccc(C(C)C)c1NC(=O)N(C)C(C)c1ccccc1]

L7 was prepared following a modified literature procedure.²⁵ In particular, 2,6-diisopropylaniline (1.32 g, 7.40 mmol) was dissolved in dichloromethane and the solution was cooled to 0° C. Triphosgene (724 mg, 2.44 mmol) was added in portions as a solid. The solution was stirred for five minutes after which N,N-diisopropylethylamine (DIPEA; 1.91 g, 14.8 mmol) was added and the cold bath removed. The solution was stirred for 1 hour and then N-methyl-1-phenylethan-1-amine (1.00 g, 7.40 mmol) and a second portion of DIPEA (0.96 g, 7.4 mmol) was added. The solution was stirred for an additional hour, and then diluted with 1M HCl. The organic phase was washed three times with 1M HCl, dried over MgSO₄, filtered, and concentrated by rotary evaporation to give the crude product. Recrystallization from a concentrated ethyl acetate solution provided the product L7 as a white solid (1.81 g, 5.34 mmol, 72%). ¹H NMR (CDCl₃, 400 MHz, rt): δ=1.31 (s, 12H, CH—(CH₃)₂), 1.72 (s, 3H, CH—CH₃), 3.00 (s, 3H, N—CH₃), 3.22-3.12 (m, 2H, CH—(CH₃)₂), 5.78-5.72 (m, 2H, CH—CH₃, NH), 7.28 (m, 1H, Ar—CH), 7.37-7.35 (m, 1H, Ar—CH), 7.45-7.39 (m, 2H, Ar—CH), 7.51-7.50 (m, 4H, Ar—CH) ppm. ¹³C NMR (CDCl₃, 101 MHz, rt): δ=17.34 (CH—CH₃), 23.81 (CH—(CH₃)₂), 28.79 (CH—(CH₃)₂), 29.82 (N—CH₃), 52.99 (N—CH), 123.36, 126.95, 127.41, 127.63, 128.73, 132.80, 142.12, 146.52 (Ar—C, Ar—CH), 157.22 (C═O) ppm. HRMS (ESI): m/z calc. for C₂₂H₃₁N₂O [M+H⁺]: 339.2437. Found: 339.2444. EA: calc. for C₂₂H₃₀N₂O: C, 78.06; H, 8.93; N, 8.28. Found: C, 78.18; H, 8.96; N, 8.31.

### (vii) 2, 3-bis(2,6-diisopropylphenyl)-1,1-diisopropylguanidine (L8)

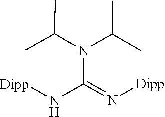

L8 was prepared following a literature procedure.²⁶ The chemical shifts for the title compound matched those previously reported.

### (b) Titanium Complexes

### (i) Tetrakis(trimethylsilylmethyl)titanium (Ti2)

Ti(CH₂SiMe₃)₄

Ti2 was prepared following a literature procedure.²⁷ The chemical shifts for the title compound matched those previously reported.

### (ii) Bis(3-(2,6-diisopropylphenyl)-1-methyl-1-(1-phenylethyl)ureate)bis(dimethylamido)titanium (Ti3)

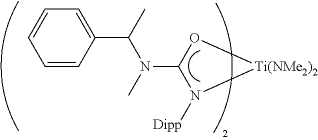

L7 (100.0 mg, 0.296 mmol) was dissolved in 5 ml n-hexane and Ti(NMe₂)₄ (33.1 mg, 0.148 mmol) was added with a micropipette. The reaction mixture was stirred for 16 h at room temperature. The solvent was removed in vacuo and Ti3 was isolated as orange solid (108 mg, 0.133 mmol, 90%) without any further purification. Single crystals suitable for X-ray crystallography were grown from a saturated n-hexane solution at room temperature. ¹H NMR (toluene-d₈, 400 MHz, rt): δ=1.11-1.43 (m, 32H), 1.97-2.00 (m, 4H), 3.20 (s, 9H), 3.23 (s, 3H), 3.62-3.88 (m, 4H), 6.12-6.24 (m, 2H), 7.02-7.14 (m, 8H), 7.17-7.27 (m, 4H), 7.37-7.50 (m, 4H) ppm. A ¹³C NMR spectrum in high resolution was not obtained, due to poor solubility in C₆D₆ and toluene-d₈ and additional coalescence effects at room temperature.

### III. General Hydroaminoalkylation Reaction Procedures

(a) Ligand screening; General procedure: The ligand (0.050 mmol) was weighed into a vial and dissolved with toluene-d₈ (0.3 mL). Ti(NMe₂)₄/Ti(CH₂SiMe₃)₄(0.025 mmol) was added with a micropipette. 4-Methoxy-N-methylaniline (0.5 mmol) and norbornene (0.5 mmol) were weighed into a different vial, dissolved with toluene-d₈ (0.2 mL) and added to the catalyst system with a micropipette. The resultant reaction mixture was transferred into a J. Young NMR tube and the vials were rinsed with an additional 0.2 mL of toluene-d₈. An initial ¹H NMR spectrum was recorded, and the sample was added to a preheated oil bath. All conversion values were determined by ¹H NMR spectroscopy.

(b) Comparison of the reactivity of in situ catalyst system generated using ligands L5 and L7 and the pre-isolated catalyst system Ti3; General procedure: The ligand (0.125 mmol) was weighed into a vial and dissolved with toluene-d₈ (1.5 mL). Ti(NMe₂)₄ (0.125 mmol) added with a micropipette. N-methylaniline (2.5 mmol), 1-octene (2.5 mmol) and trimethoxybenzene (140 mg, 0.835 mmol) were weighed into a different vial, dissolved with toluene-d₈ (2.0 mL) and added to the catalyst system with a micropipette. The resultant reaction mixture was split into 5 identical volumes and these were again transferred into 5 J. Young NMR tubes. For the catalyst system Ti3 the first two steps were combined. Initial ¹H NMR spectra were recorded, the samples were added to a preheated oil bath and individually collected after the corresponding times. All yields were determined by ¹H NMR spectroscopy.

(c) Investigations of amine and alkene scope using ligand L7; General procedure: L7 (8.5 mg, 0.025 mmol) was weighed into a vial and dissolved with toluene-d₈ (0.3 mL). Ti(NMe₂)₄(5.6 mg, 0.025 mmol) was added with a micropipette. The selected amine (0.5 mmol), alkene (0.5 mmol) and trimethoxybenzene (28.0 mg, 0.166 mmol) were weighed into a different vial, dissolved with toluene-d₈ (0.2 mL) and added to the catalyst system with a micropipette. The resultant reaction mixture was transferred into a J. Young NMR tube and the vials were rinsed with an additional 0.2 mL of toluene-d₈. An initial ¹H NMR spectrum was recorded, and the sample was added to a preheated oil bath. All yields were determined by ¹H NMR spectroscopy and GC analysis. The selectivity was determined by GC analysis (product area shown).

(d) Reaction vessel comparison; General procedure: L7 (17.0 mg, 0.05 mmol) was weighed into the selected reaction vessel and dissolved with 1-octene (281 mg, 2.5 mmol) as solvent. Ti(NMe₂)₄(11.2 mg, 0.05 mmol) and N-methylaniline (107 mg, 1.0 mmol) were added with a micropipette. The resultant reaction mixture was heated for 2 hours or until completion of the reaction. All conversions were determined by GC analysis and compared to the results of III(b) and III(c). Experiments in closed systems should only be run in reaction vessels that can withstand the autogenous pressure and in a closed fume hood.

For Method D reactions with up to 5 mmol of alkene are feasible, however the condensation of alkene can be observed in the syringe part of the pressure relief. Consequently, extremely long reaction times over multiple days with specific substrates should be avoided.
Method A: 8 mL High pressure glass vial: 64% (2 h), completion after 4 h.
Method B: 100 mL Schlenk flask with Teflon stopper: Completion after 4 h.
Method C: 100 mL Schlenk flask with balloon: Completion after 3 h.
Method D: 8 mL Standard glass vial with balloon: Completion after 4 h.

### IV. Synthetic Procedures for Application Examples

### (a) N-((bicyclo[2.2.1]hept-5-en-2-yl)methyl)aniline (1)

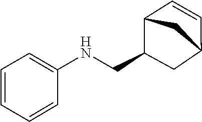 [SMILES: C1=C[C@@H]2C[C@H]1C[C@H]2CNc1ccccc1]

L7 (8.5 mg, 0.025 mmol) was weighed into a high pressure vial, dissolved in norbornadiene (460 mg, 5.0 mmol) and Ti(NMe₂)₄ (5.6 mg, 0.025 mmol) was added with a micropipette. After that, N-methylaniline (54.0 mg, 0.5 mmol) was weighed into the same vial. The vial was placed into a preheated aluminium block at 160° C. for 8 h. The crude product was filtered through Celite™ and the residual norbomadiene (NBD) was removed in vacuo (NBD could be reused). No further purification steps were needed for further use. The product 1 was isolated as a yellow oil (98.0 mg, 0.5 mmol, quant.). The chemical shifts for the title compound matched those previously reported.²⁸

### (b) N-(cyclooct-4-en-1-ylmethyl)aniline (2)

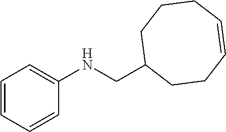 [SMILES: C1=C\CCC(CNc2ccccc2)CCC/1]

L7 (17.0 mg, 0.05 mmol) was weighed into a high pressure vial, dissolved in cyclooctadiene (270 mg, 2.5 mmol) and Ti(NMe₂)₄ (11.2 mg, 0.05 mmol) was added with a micropipette. After that, N-methylaniline (54.0 mg, 0.5 mmol) was weighed into the same vial. The vial was placed into a preheated aluminium block at 160° C. for 72 h. The crude product was filtered through Celite and the residual cyclooctadiene (COD) was removed in vacuo (COD could be reused). No further purification steps were needed for further use. The product 2 was isolated as a colorless oil (107.0 mg, 0.5 mmol, quant.). The chemical shifts for the title compound matched those previously reported.²³

### (c) N-(4-((^{t}butyldimethylsilyl)oxy)-2-methylbutyl)-4-methoxyaniline (3)

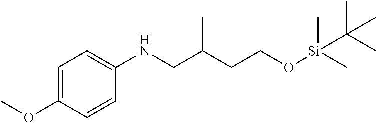 [SMILES: COc1ccc(NCC(C)CCO[Si](C)(C)C(C)(C)C)cc1]

L7 (123 mg, 0.36 mmol) was weighed into a vial, dissolved in 5 mL toluene and Ti(NMe₂)₄(82 mg, 0.36 mmol) was added with a micropipette. After that, 4-methoxy-N-methylaniline (500 mg, 3.6 mmol) and (but-3-en-1-yloxy)(tert-butyl)dimethylsilane (680 mg, 3.6 mmol) were weighed into the same vial. The resultant reaction mixture was transferred into a Schlenk flask. The flask was placed into a preheated oil bath at 160° C. for 12 h. The crude product was purified by chromatography (SiO₂, pentane/EA=15:1) and after removal of all volatiles in vacuo, the product 3 was isolated as a colorless oil (1106 mg, 3.4 mmol, 94%). ¹H NMR (CDCl₃, 400 MHz, rt): δ=0.06 (s, 6H, Si—(CH₃)₂), 0.90 (s, 9H, C—(CH₃)₃), 0.99 (d, J=6.7 Hz, 3 H, CH—CH₃), 1.18-1.20 (m, 1H), 1.37-1.48 (m, 1H), 1.58-1.72 (m, 1H), 1.84-2.00 (m, 1H), 2.88-3.06 (m, 2H), 3.55 (br. s, 1H), 3.60-3.71 (m, 1H), 3.75 (s, 3H, O—CH₃), 6.55-6.58 (m, 2H, Ar—H), 6.76-6.79 (m, 2H, Ar—H) ppm. ¹³C NMR (CDCl₃, 101 MHz, rt): δ=−5.14 (Si—(CH₃)₂), 18.43 (CH—CH₃), 26.11 (C—(CH₃)₃), 30.01 (CH—CH₃), 37.97, 51.47, 56.02, 61.24, 114.02 (Ar—CH), 115.07 (Ar—CH), 143.09 (Ar—C), 151.93 (Ar—C) ppm. HRMS (ESI): m/z calc. for C₁₈H₃₄NO₂Si[M+H⁺]: 324.2359. Found: 324.2356.

### (d) 4-((4-methoxyphenyl)amino)-3-methylbutan-1-ol (4)

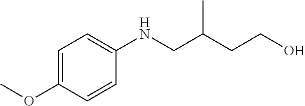

4 was prepared following a modified literature procedure.²⁹ In particular, 3 (250 mg, 0.77 mmol) was dissolved in 10 mL tetrahydrofuran (THF) and 1M tetra-n-butylammonium fluoride (TBAF; 0.85 mL, 0.85 mmol) was added. The reaction mixture was stirred for 16 h at room temperature, diluted with water, extracted with ethyl acetate, washed with brine and dried over MgSO₄. After removal of all volatiles in vacuo, the product 4 was isolated as a yellow oil (153 mg, 0.73 mmol, 95%). ¹H NMR (CDCl₃, 400 MHz, rt): δ=1.00 (d, J=6.8 Hz, 3 H, CH—CH₃), 1.46-1.57 (m, 1H), 1.63-1.74 (m, 1H), 1.83-1.92 (m, 1H, CH—CH₃), 2.90-3.02 (m, 4H), 3.62-3.72 (m, 2H), 3.74 (s, 3H, O—CH₃), 6.60-6.63 (m, 2H, Ar—H), 6.77-6.80 (m, 2H, Ar—H) ppm. 13C NMR (CDCl₃, 101 MHz, rt): δ=18.80 (CH—CH₃), 31.00 (CH—CH₃), 38.38, 52.04, 55.93 (0-CH₃), 61.02, 114.88 (Ar—CH), 115.02 (Ar—CH), 142.54 (Ar—C), 152.54 (Ar—C) ppm. HRMS (ESI): m/z calc. for C₁₂H₂₀NO₂[M+H⁺]: 210.1494. Found: 210.1488.

### (e) 4-((tert-Butoxycarbonyl)(4-methoxyphenyl)amino)-3-methylbutanoic acid (5)

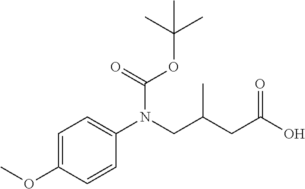 [SMILES: COc1ccc(N(CC(C)CC(=O)O)C(=O)OC(C)(C)C)cc1]

4 (100 mg, 0.48 mmol) was dissolved in 7 mL dichloromethane (DCM) and triethylamine (59 mg, 0.58 mmol) was added. The reaction mixture was cooled to 0° C. under argon atmosphere and di-tert-butyl dicarbonate (126 mg, 0.58 mmol) in 3 mL DCM was added. After 1 h at 0° C., the reaction mixture was stirred for another 16 h at room temperature. The mixture was then washed sequentially with brine, saturated citric acid solution, saturated sodium hydrogen carbonate solution and water. The organic layer was dried over MgSO₄ after removal of all volatiles in vacuo, and the protected amino alcohol (142 mg, 0.46 mmol, 96%) was directly oxidized without further purification. 5 was prepared following a modified literature procedure.³⁰ A solution of protected amino alcohol (142 mg, 0.46 mmol) in 5 mL tert-butyl alcohol was treated with 0.5 M NaOH (3.68 mL, 1.84 mmol) and 10% KMnO₄ solution in water (2.91 mL, 1.84 mmol). The reaction mixture was stirred for 16 h at room temperature and after quenching with an excess of 5% sodium thiosulfate solution in water, the mixture was washed with diethyl ether and the aqueous phase was acidified to a pH of 1-2 with 1 M HCl. After extraction with ethyl acetate, the combined organic phases were dried over MgSO₄. After removal of all volatiles in vacuo, the product 5 was isolated as a colorless solid (152 mg, 0.47 mmol, 84%). ¹H NMR (CD₃OD, 400 MHz, rt): δ=0.95 (d, J=6.4 Hz, 3 H, CH—CH₃), 1.41 (s, 9H, C—(CH₃)₃), 2.02-2.08 (m, 2H), 2.37-2.39 (m, 1H), 3.48-3.60 (m, 2H), 3.80 (s, 3H, O—CH₃), 6.90-6.92 (m, 2H, Ar—CH), 7.11-7.14 (m, 2H, Ar—CH) ppm. 13C NMR (CD₃OD, 101 MHz, rt): δ=17.85 (CH—CH₃), 28.63 (C—(CH₃)₃), 30.85, 39.83, 55.91 (0-CH₃), 56.35, 115.19, 129.67, 136.23, 157.25 (N—CO), 159.64, 176.36 (OC—OH) ppm. (C—(CH₃)₃ very broad and not listed). HRMS (ESI): m/z calc. for C₁₇H₂₅NNaO₅[M+Na⁺]: 346.1630. Found: 346.1632.

### (f) 1-(4-methoxyphenyl)-3-methylpyrrolidine (6)

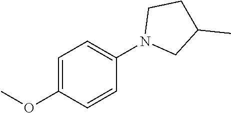

6 was prepared following a modified literature procedure.³¹ In particular, 3 (250 mg, 0.77 mmol) was dissolved in 5 mL acetonitrile and tosyl fluoride (404 mg, 2.32 mmol) and diazabicyclo[5.4.0]undec-7-ene (DBU; 353 mg, 2.32 mmol) were added. The reaction mixture was heated to 90° C. for 48 h. The solution was cooled, diluted with 1M NaOH (10 mL), stirred at room temperature for 1 hour and diluted with ethyl acetate (20 mL). The organic phase was then washed with water and brine, dried over MgSO₄ and concentrated in vacuo. The crude product was purified by chromatography (SiO₂, pentane/EA=20:1) and after removal of all volatiles in vacuo, the product 6 was isolated as a yellow oil (100 mg, 0.52 mmol, 68%). The chemical shifts for the title compound matched those previously reported.²⁹

### (g) 3-methyl-pyrrolidine hydrochloride (7)

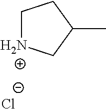

6 (40 mg, 0.21 mmol) was dissolved in 5 mL acetonitrile/water (1:1) and trichloroisocyanuric acid (24 mg, 0.11 mmol) and 1 M H₂SO₄ (0.21 mL). The reaction mixture was heated to 90° C. for 30 min and after completion, as determined by thin layer chromatography (TLC), the mixture was washed with DCM. The aqueous phase was subsequently brought to pH 13-14 through addition of 5 M aqueous NaOH and washed with ethyl acetate to remove the remaining impurities (3-methyl-pyrrolidine is easily soluble in water, even under basic conditions). The aqueous phase was subsequently brought back to pH 0-1 via addition of an excess of 1M aqueous HCl and lyophilized overnight to afford a brown solid (product/NaCl mixture). After filtration, and washing with an excess of DCM, all volatiles were removed in vacuo and the product 7 was isolated as a brown oil (23 mg, 0.19 mmol, 91%). The ¹H chemical shifts for the title compound correlated to those previously reported of the free amine.^{32 1}H NMR (CD₃OD, 400 MHz, rt): δ=1.17 (d, J=6.7 Hz, 3 H, CH—CH₃), 1.57-1.70 (m, 1H), 2.16-2.27 (m, 1H), 2.35-2.52 (m, 1H, CH—CH₃), 2.76-2.83 (m, 1H), 3.23-3.48 (m, 3H) ppm. 13C NMR (CD₃OD, 101 MHz, rt): δ=17.18 (CH—CH₃), 33.15, 34.10 (CH—CH₃), 46.42, 52.71 ppm. HRMS (ESI): m/z calc. for C₅H₁₂N [M+H⁺]: 86.0970. Found: 86.0974.

### B. Results and Discussion

Ligand Design and Screening. A rapid ligand screening procedure was employed for probing a diverse set of 1,3-N,X-chelating (X=N or O) ligand effects in hydroaminoalkylation catalysis. The reactive catalyst system was generated in situ (Table 1) via a protonolysis reaction of 2 equivalents of ligand to 1 equivalent of metal precursor to study their activity in the hydroaminoalkylation reaction between norbomene and 4-methoxy-N-methylaniline in toluene-d₈ at 160° C. for 24 h. The conversions to the hydroaminoalkylation products were determined by integration of the ortho-protons of the aromatic amines. The substrates norbomene and 4-methoxy-N-methylaniline were specifically chosen as a starting point for the present investigations, because of their general high reactivity in the hydroaminoalkylation (such as inductive effects and ring strain). For the titanium source, the widely commercially available and relatively inexpensive Ti(NMe₂)₄ (Ti1) and, the relatively easy to synthesize Ti(CH₂SiMe₃)₄ (Ti2) were used. Neat Ti2 has been described as decomposing under room light and deteriorating within hours at room temperature.³³ However, we observed that it can be handled as a solution in the glovebox for several hours without excluding ambient light and we did not observe any reactivity decline after storage overnight at room temperature. Consequently, the handling of this titanium catalyst precursor has a notable advantage over the highly reactive TiBn₄, which has to be stored indefinitely at −30° C.

TABLE 1
Benchmark hydroaminoalkylation reaction of norbornene and 4-methoxy-N-
methylaniline for in situ ligand screening for Ti1 and Ti2ª

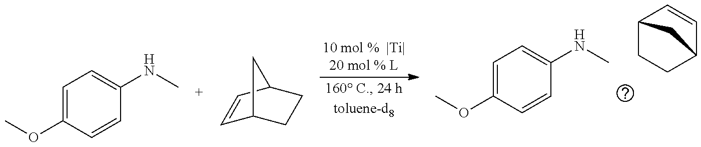

| | | Ti(NMe₂)₄ | Ti(CH₂SiMe₃)₄ |
|---|---|---|---|
| | | (Ti1) | (Ti2) |
| | — | 78% (96 h) | 16% |
| L1 | 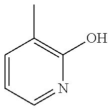 | 23% | 10% |
| L2 | 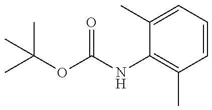 [SMILES: Cc1cccc(C)c1NC(=O)OC(C)(C)C] | 0% | 0% |
| L3 | 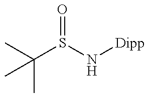 | 0% | 0% |
| L4 | 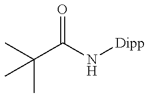 | 90% | 74% |
| L5 | 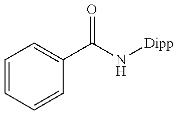 | 97% | 86% |
| L6 | 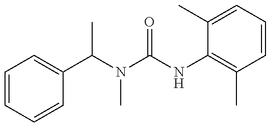 [SMILES: Cc1cccc(C)c1NC(=O)N(C)C(C)c1ccccc1] | 92% | 91% |
| L7 | 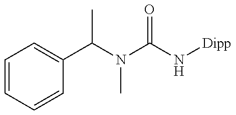 | 100% | 100% |
| L8 | 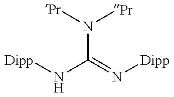 | 33% | 35% |
| L9 | 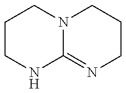 | 0% | 0% |

^{a}Reaction conditions: 4-Methoxy-N-methylaniline (0.5 mmol), norbornene (0.5 mmol), Ti1/Ti2 (0.025 mmol), ligand (0.05 mmol), toluene-d₈ (0.7 mL). Conversion determined by ¹H NMR spectroscopy. All data presented have been collected in duplicate or triplicate.

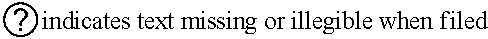

First, the homoleptic titanium sources were tested without any addition of supporting ligands. Ti(NMe₂)₄(Ti1) is one of the first tested catalysts for hydroaminoalkylation and it is known for its low reactivity even with activated substrates (78% after 96 h).³⁴ Ti(CH₂SiMe₃)₄ (Ti) showed similar low activity under the conditions used (16% after 24 h). Further investigations with the use of supporting ligands were not carried out with Ti2 as the titanium source as the conversions were comparable to Tit or lower. Additionally, the formation of polynorbornene was observed in several cases, which, while not wishing to be limited by theory, is likely attributed to the formation of titanium carbene complexes via α-H-abstraction of the CH₂SiMe₃-unit that catalyzes ring-opening metathesis polymerization (ROMP) of norbomene. Consequently, the remainder of the studies focused on the commercially available Ti(NMe₂)₄(Ti1).

The use of pyridonates for tantalum catalyzed intermolecular hydroaminoalkylation³ and pyridonate titanium complexes in accessing intramolecular hydroaminoalkylation over hydroamination has been previously reported.³⁶ In both cases, 2-substituted pyridonate ligands showed good activity for this specific transformation and especially 3-methylpyridin-2-ol (L1) was demonstrated to be an excellent candidate for the intermolecular catalysis. However, for the reaction of norbomene with 4-methoxy-N-methylaniline and Ti1 as the titanium source, L1 as the ligand produced only small amounts of product (23%).

The use of carbamate L2 and sulfinamide L3 was also studied. Even though the latter belongs to a class of ligands that are already known to be versatile ligands in other enantioselective catalytic transformations³⁷, to our knowledge, they have not been used in early-transition metal catalyzed hydrofunctionalizations. However, both ligand classes were very susceptible to nucleophilic attack, especially under the present Lewis-acidic conditions at high temperatures, and consequently no conversion for the hydroaminoalkylation reaction was observed. Additionally, it was not possible to identify or isolate L2 and L3 after the reaction, which is further evidence of the reaction between the ligands and the hydroaminoalkylation substrates.

The amidate ligands showed that relatively large substituents on the nitrogen site of the ligand (>isopropyl or phenyl) were desirable for reactivity. The most active amidate ligands were L4 and L5 with 90% and 97% conversion, which exhibit a different substitution pattern (tert-butyl vs. phenyl) on the carbonyl site of the ligand but have the same bulky ligand on the nitrogen. Other ligands for the hydroaminoalkylation of norbomene with 4-methoxy-N-methylaniline which gave conversion but at lower levels than L4 or L5 are shown in Scheme 1.

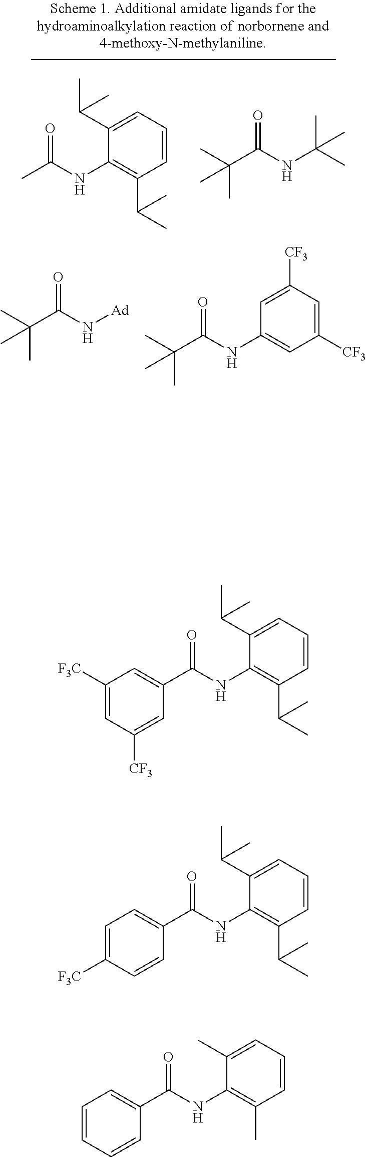

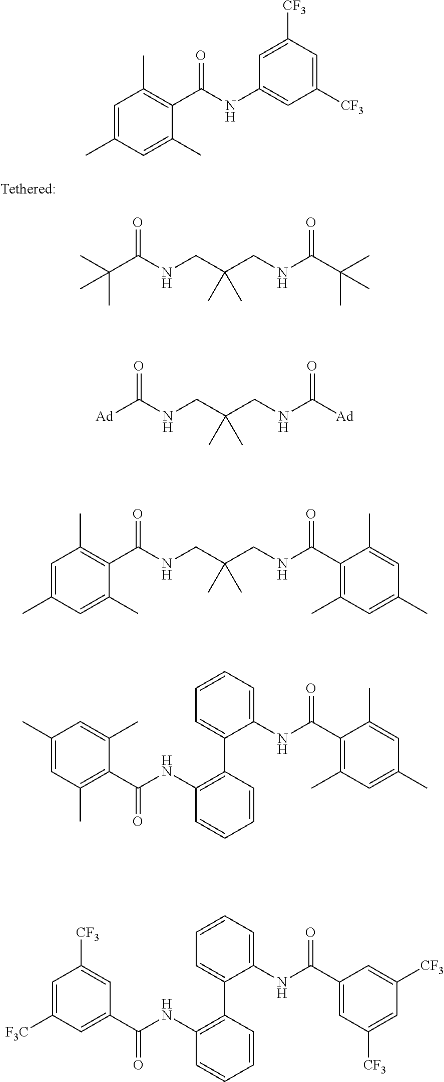

The range of conversion for ureate ligands was generally narrower and more consistent (e.g. starting from >50% except for tethered ligands which showed lower values for conversion) in comparison to the amidate ligands with the acyclic ureates demonstrating slightly higher reactivity over the cyclic counterparts. L6 was further sterically enhanced to achieve in L7 the best observed conversion value for this reactivity screening (92% for L6 to quantitative for L7). Other ligands for the hydroaminoalkylation of norbomene with 4-methoxy-N-methylaniline which gave conversion but at lower levels than L6 or L7 are shown in Scheme 2.

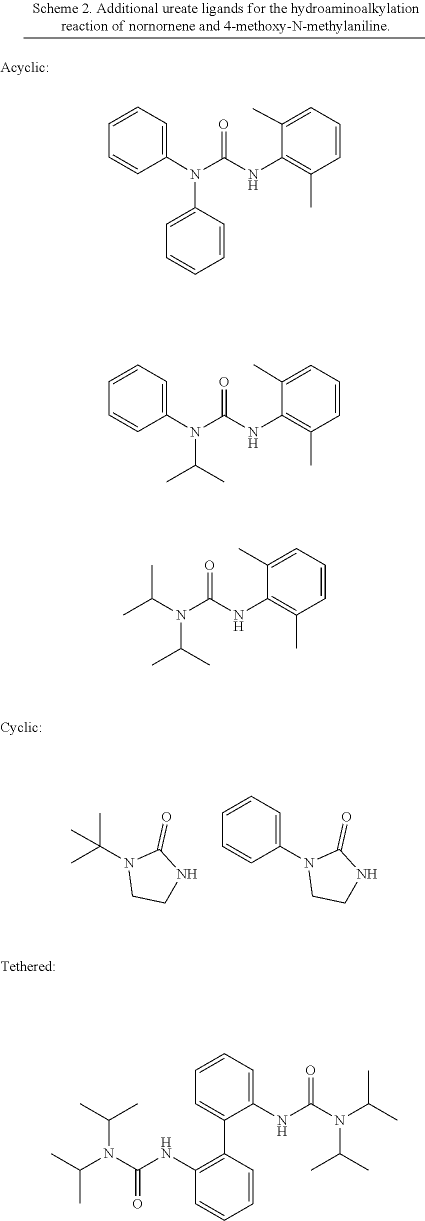

Finally, in contrast to previous work on aminopyridinate and formamidinate ligands³⁸, which demonstrated highly active systems formed with specific substitution patterns on the 1,3-N,N-chelating ligand class the guanidines L8 and L9 had no or low (0-33%) conversion. In the case of L8, this was attributed to reaction of ligand material with the excess of 4-methoxy-N-methylaniline under Lewis-acidic conditions to form the corresponding guanidine, which could be isolated after the screening. This process inhibits the hydroaminoalkylation reactions progression, therefore L8 and L9 were deemed non-suitable.

Reactivity Studies. With the identification of L5 and L7 as candidates for further reactivity studies, the above-described benchmark reaction was repeated with 5% and 10% catalyst loading, which is common for this type of early-transition metal hydrofunctionalizations, and varied the equivalents of ligand (1 eq., 2 eq., and 3 eq. with respect to catalyst loading) to investigate the best ligand option and loading. Additionally, N-methylaniline and unactivated terminal olefin 1-octene were investigated as more challenging substrates and the catalyst parameters reduced to a minimal loading of 5% for the titanium precatalyst and 5% for the ligand. Furthermore, instead of waiting for the completion of the reaction, multiple early datapoints in the presence of 1,3,5-trimethoxybenzene as internal standard were collected (NMR yields).

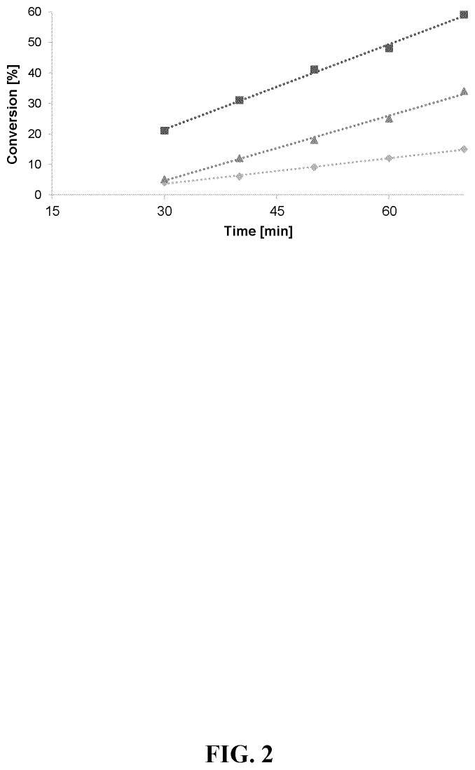

For both ligands L5 and L7 linear behavior for the reaction progression over time in the selected time area from 30 to 70 minutes was observed (FIG. 2, Table 2).

TABLE 2
Reactivity Comparison of L5, L7 and Ti3*

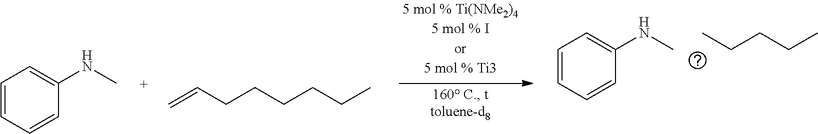

| Time (min) | Yield for L5 | Yield for L7 | Yield for Ti3 |
|---|---|---|---|
| 30 | 4% | 21% | 5% |
| 40 | 6% | 31% | 12% |
| 50 | 9% | 41% | 18% |
| 60 | 12% | 48% | 25% |
| 70 | 15% | 59% | 34% |

*Reaction conditions: N-Methylaniline (0.5 mmol), 1-octene (0.5 mmol), toluene-d₈ (0.7 mL), Ti(NMe₂)₄ (0.025 mmol), ligand (0.025 mmol) or Ti3 (0.025 mmol). Yields determined by ¹H NMR spectroscopy. All data presented have been collected in duplicate or triplicate.

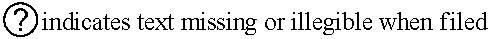

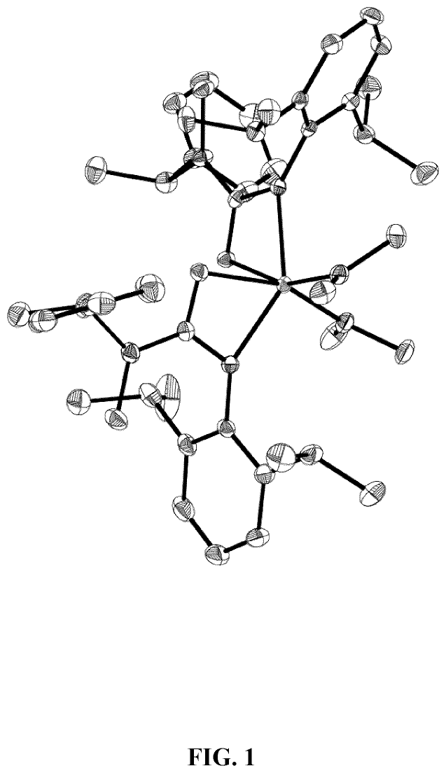

Consequently, simple linear regression was applied in these cases to determine the initial turn over frequencies (TOF) for both systems. Whereas for the reaction of norbomene with 4-methoxy-N-methylaniline over 24 h the amidate L5 and ureate L7 seemed to be interchangeable, the 11 h⁻¹ TOF for L7 for this more challenging reaction proved to be superior over the less reactive L5 with a TOF of only 3 h⁻¹. This suggests that ureate ligands enhance the electrophilic character of the titanium center to a better degree than the comparable amidate ligands. Additionally, the system with L7 demonstrates superior TOFs for this specific reaction with unactivated substrates in direct comparison to other titanium catalysts based on commercially available titanium amido precursors and does not show any indication of interference of the liberated dimethylamine for the hydroaminoalkylation process, which is observed in other systems.³⁹ Furthermore, a direct comparison of this in situ system to the pre-isolated titanium catalyst was of interest. However, even when cooled down to −30° C. and with slow addition of the ligand to a very dilute solution of Ti(NMe₂)₄ (Ti1), only a mixture of mono- and bisligated titanium ureate complexes could be isolated. This mixture further disproportionated to Ti(NMe₂)₄(Ti1) and bisligated titanium ureate complex Ti3 at room temperature overnight. However, Ti3 can also be selectively synthetized and characterized by single-crystal X-ray diffraction (FIG. 1, Table 3).

TABLE 3
Crystal Structure Data of Ti3.

| Empirical formula | C₄₈H₇₀N₆O₂Ti | |
|---|---|---|
| fw | 811.00 | |
| Color | Orange | |
| Crystal dimension, mm | 0.37 × 0.287 × 0.09 | |
| Crystal system | triclinic | |
| Space group | P-1 | |
| a, Å | 11.006 | (2) |
| b, Å | 14.517 | (3) |
| c, Å | 16.170 | (3) |
| α, deg | 67.282 | (5) |
| β, deg | 77.986 | (6) |
| γ, deg | 89.059 | (6) |
| V, Å³ | 2325.0 | (8) |
| Z | 2 | |
| D_{caclcd}, g cm⁻³ | 1.158 | |
| μ, mm⁻¹ | 0.227 | |
| T, K | 273.15 | |
| 2θ range, deg | 3.05-56.71 | |
| No. of rflns collected | 52373 | |
| No. of indep rflns (R(int)) | 11577 | (0.0357) |

| | |
|---|---|
| Final R indices [I > 2δ(I)] | R₁ = 0.0413, wR₂ = 0.0993 |
| R indices (all data) | R₁ = 0.0544, wR₂ = 0.1061 |
| GOF on F² | 1.019 |
| Largest diff peak/hole, e Å⁻³ | 0.99/−0.35 |

Surprisingly, Ti3 shows only slightly lower initial TOFs for the reaction of N-methylaniline and 1-octene (Table 2, FIG. 2, 9 h⁻¹), but starts with a significantly lower conversion for the observed time area from 30 to 70 min. While not wishing to be limited by theory, this indicates that the additional ligand not only slightly interferes with the hydroaminoalkylation mechanism by blocking one of the coordination sites for incoming substrates, but also that the additional ligand has to be exchanged by amine substrates at higher temperatures (induction period) to achieve similar reactivity than the in situ system. Consequently, while not wishing to be limited by theory, this could further hint towards a bimetallic mechanism with bridged species.

Scope of Alkenes and Amines. The alkene substrate scope of the catalyst system prepared with Ti1 and L7 was explored for N-methylaniline and terminal alkenes (Table 4, Entries 1-7) using a similar NMR screening technique as for the reactivity screening.

TABLE 4
Scope of system Ti1 and L7 for hydroaminoalkylation of various alkenes with
N-methylaniline*

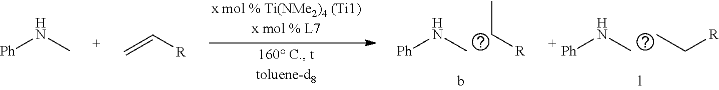

| | | Catalyst | | |
|---|---|---|---|---|
| | | Loading | Yield | Selectivity |
| Entry | Alkene | Time | (b + 1) | (b/1) |
| 1 | 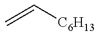 | 5% 2 h | 95% | >99:1 |
| 2 | 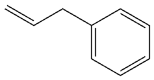 | 5% 6 h | 96% | 94:6 |
| 3 | 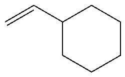 | 5% 8 h | 97% | >99:1 |
| 4 | 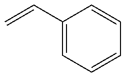 | 5% 8 h | 96% | 63:37 |
| 5 | 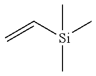 | 10% 16 h | 99% | 61:39 |
| 6 | 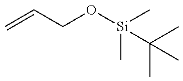 | 20% 48 h | 71% | >99:1 |
| 7 | 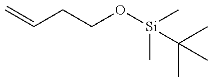 | 5% 2 h | 96% | 99:1 |

*Reaction conditions: N-methylaniline (0.5 mmol), alkene (0.5 mmol), toluene-d₈ (0.7 mL), catalyst loadings as shown. Yields determined by ¹H NMR spectroscopy and GC analysis. Selectivity was determined by GC analysis. All data presented have been collected in duplicate or triplicate.

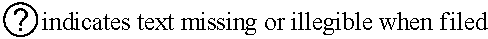

Reaction times were adapted to favor full conversions of substrate while maintaining minimal catalyst loading. In line with what was observed for the reactivity screening, 1-octene (Entry 1) was close to complete conversion with excellent regioselectivities for the branched product within 2 h, which is considerably faster than most of the other reported titanium catalysts based on commercially available titanium amido precursors³⁹ except for the catalyst using highly active TiBn₄ as precursor under neat conditions.⁴⁰ Interestingly, Ti1 and L7 can be used in loadings of 1% with similar results for these specific reactions, however the reaction times increase exponentially to 72 h for full conversion. With increase of steric bulk on the alkene site from benzyl to cyclohexyl and phenyl (Entries 2-4), the reaction times increase slightly, but all reactions were still completed within one workday (i.e. less than 8 h). Surprisingly, the change from cyclohexyl to phenyl and the corresponding modified electronic environment does not affect the reaction times in any regard. However, the selectivity for the branched product declines significantly. While not wishing to be limited by theory, this is attributed to the inverted electronic characteristics of styrenes in comparison to alkyl substituted alkenes, which normally prefer the formation of the linear hydroaminoalkylation products. The same behavior could be observed for trimethylvinylsilane (Entry 5, b/l=61:39). Alkenes with silyl protected alcohols, which are interesting substrates for post-hydroaminoalkylation functionalization to generate N-containing fine chemicals may also be used as a substrate. However, the allyl substitution pattern (Entry 6) increases the reaction time and catalyst loading dramatically (48 h, 20%) and complete conversion was not achieved (71%). Though, this behavior is primarily caused by the strong electronic influence of this substitution for the insertion step of the alkene. Accordingly, by adding one more carbon atom to the alkyl chain of the silyl protected alcohol (Entry 7), similar results for the reaction time and regioselectivity as for 1-octene were observed.

The amine substrate scope with the aforementioned system was also investigated (Table 5). para-substituted N-methylaniline derivatives (Entries 1 and 2) were well tolerated, including halide substituents, which are compatible with d° titanium that does normally not engage in oxidative addition/reductive elimination-type chemistry. Such aryl halides can be used in further cross-coupling reactions with late-transition metals like palladium.

TABLE 5
Scope of system Ti1 and L7 for hydroaminoalkylation of 1-octene with
various amines.*

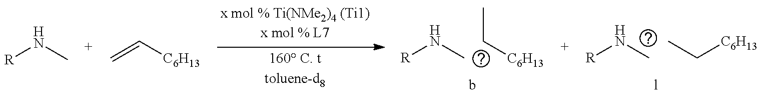

| | | Catalyst Loading | | Selectivity |
|---|---|---|---|---|
| Entry | Amine | Time | Yield (B + L) | (B/L) |
| 1 | 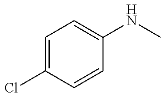 | 5% 2 h | 96% | >99:1 |
| 2 | 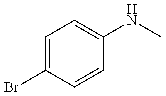 | 5% 2 h | 97% | 99:1 |
| 3 | 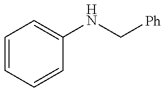 [SMILES: c1ccc(CNc2ccccc2)cc1] | 5% 16 h | 80% | 83:17 |
| 4 | 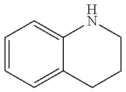 | 10% 16 h | 89% | 91:9 |
| 5 | 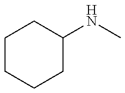 | 5% 24 h | 86% | >99:1 |
| 6 | 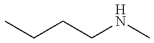 | 10% 24 h | 98% | 95:5 |

*Reaction conditions: Amine (0.5 mmol), 1-octene (0.5 mmol), toluene-d₈ (0.7 mL), catalyst loadings as shown. Yields determined by ¹H NMR spectroscopy and GC analysis. Selectivity was determined by GC analysis. All data presented have been collected in duplicate or triplicate.

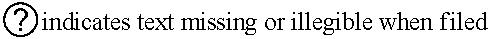

For the more sterically demanding N-benzylaniline and 1,2,3,4-tetrahydroquinoline (Entries 3 and 4, respectively) the reaction times were both increased to 16 h and for 1,2,3,4-tetrahydroqulnoline the catalyst loading had to be doubled, but in both cases good yields and only slightly lower regioselectivities were observed. The reactivity of dialkyl substituted amines was also investigated. Very challenging substrates such as N-methylcyclohexylamine and N-butylmethylamine (Entries 5 and 6) provided great yields and regioselectivity after 24 h. This is surprising, because the system using TiBn₄ could not achieve complete conversions for this substrate class due to product inhibition and deterioration of catalyst at 155° C. over longer reaction times.⁴⁰

Table 6 shows a summary of additional experimental data obtained for the hydroaminoalkylation of amines and alkenes using L7.

TABLE 6

| | | | [Ti] | [L7] | Time | Conversion | |
|---|---|---|---|---|---|---|---|
| Entry | Amine | Alkene | (mol %) | (mol %) | (h) | (%) | B:L* |
| 1 | 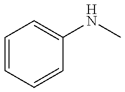 | 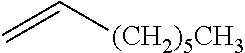 | 5 | 5 | 2 | 95 | 99:1 |
| 2 | 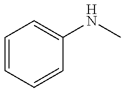 | 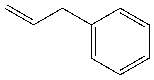 | 5 | 5 | 6 | 96 | 99:1 |
| 3 | 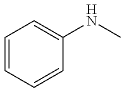 | 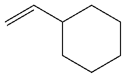 | 5 | 5 | 6 | 96 | 99:1 |
| 4ª | 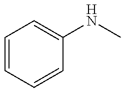 | 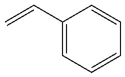 | 5 | 5 | 8 | 96 | 49:51 |
| 5 | 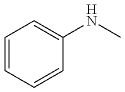 | 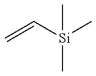 | 10 | 10 | 16 | 96 | 47:53 |
| 6 | 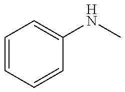 | 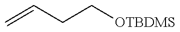 | 5 | 5 | 2 | 94 | 99:1 |
| 7 | 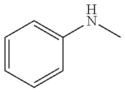 | 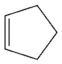 | 10 | 10 | 48 | 99 | — |
| 8 | 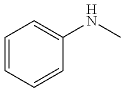 | 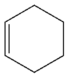 | 10 | 10 | 42 | 12 | — |
| 9 | 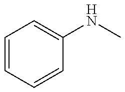 | 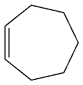 | 10 | 10 | 42 | 88 | — |
| 10 | 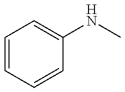 | 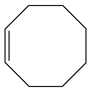 | 10 | 10 | 42 | 32 | — |
| 11^{b} | 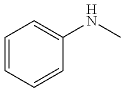 | 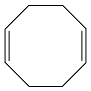 | 5 | 5 | 36 | 100 | — |
| 12 | 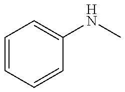 | 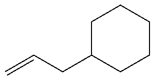 | 10 | 10 | 24 | 55(140° C.) | — |
| 13 | 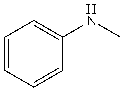 | 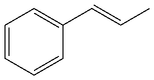 | 10 | 10 | 48 | 70 | 99:1 |
| 14 | 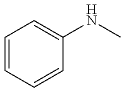 | 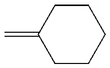 | 10 | 10 | 24 | 94 | — |
| 15 | 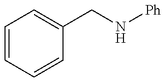 [SMILES: c1ccc(CNc2ccccc2)cc1] | 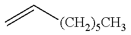 | 5 | 5 | 16 | 80 | 87:13 |
| 16^{c} | 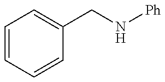 [SMILES: c1ccc(CNc2ccccc2)cc1] | 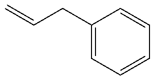 | 10 | 10 | 24 | 96 | 66:34 |
| 17 | 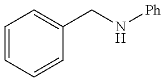 [SMILES: c1ccc(CNc2ccccc2)cc1] | 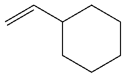 | 20 | 20 | 96 | 58 | 57:43 |
| 18ª | 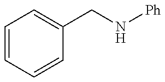 [SMILES: c1ccc(CNc2ccccc2)cc1] | 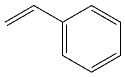 | 20 | 20 | 96 | 81 | 96:4 |
| 19 | 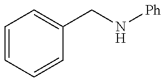 [SMILES: c1ccc(CNc2ccccc2)cc1] | 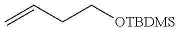 | 10 | 10 | 24 | 70 | 64:36 |
| 20 | 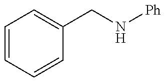 [SMILES: c1ccc(CNc2ccccc2)cc1] | 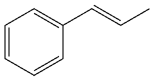 | 20 | 20 | 96 | 19 | — |
| 21 | 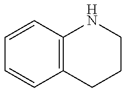 | 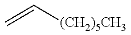 | 10 | 10 | 16 | 89 | 91:9 |
| 22 | 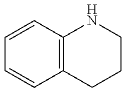 | 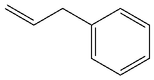 | 10 | 10 | 24 | 81 | 75:25 |
| 23 | 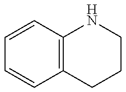 | 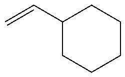 | 15 | 15 | 48 | 85 | 98:2 |
| 24ª | 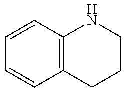 | 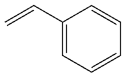 | 10 | 10 | 24 | 80 | 49:51 |
| 25 | 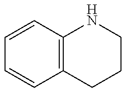 | 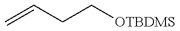 | 10 | 10 | 24 | 87 | 10:1 |
| 26 | 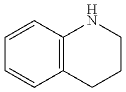 | 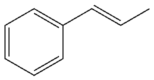 | 20 | 20 | 96 | 45 | — |
| 27 | 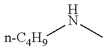 | 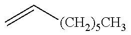 | 10 | 10 | 48 | 98 | 95:5 |
| 28 | 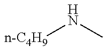 | 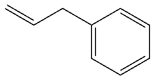 | 10 | 10 | 48 | 100 | 99:1 |
| 29 | 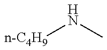 | 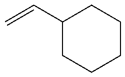 | 10 | 10 | 48 | 100 | 99:1 |
| 30ª | 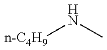 | 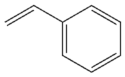 | 10 | 10 | 48 | 68 | — |
| 31 | 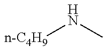 | 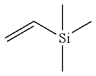 | 10 | 10 | 48 | 91 | — |
| 32 | 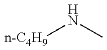 | 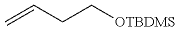 | 10 | 10 | 48 | 82 | 99:1 |
| 33 | 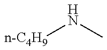 | 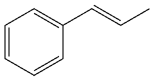 | 20 | 20 | 96 | 95 | — |
| 34 | 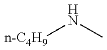 | 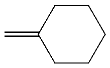 | 10 | 10 | 96 | 82 | — |
| 35 | 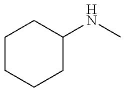 | 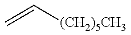 | 5 | 5 | 24 | 86 | 99:1 |
| 36 | 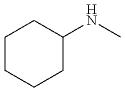 | 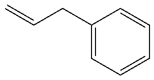 | 10 | 10 | 24 | 100 | 99:1 |
| 37 | 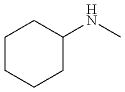 | 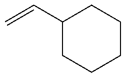 | 10 | 10 | 48 | 100 | 99:1 |
| 38ª | 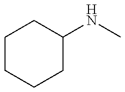 | 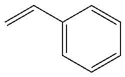 | 10 | 10 | 24 | 73 | 70:30 |
| 39 | 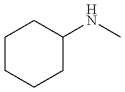 | 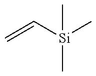 | 10 | 10 | 24 | 64 | — |
| 40 | 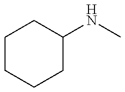 | 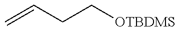 | 15 | 15 | 24 | 100 | 94:6 |
| 41 | 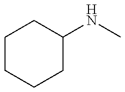 | 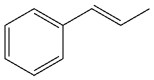 | 20 | 20 | 96 | 71 | — |
| 42 | 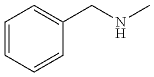 | 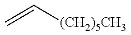 | 10 | 10 | 16 | 54 | 78:22 |
| 43 | 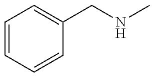 | 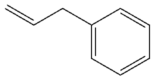 | 10 | 10 | 24 | 100 | — |
| 44 | 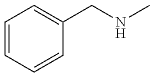 | 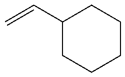 | 10 | 10 | 48 | 100 | — |
| 45ª | 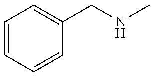 | 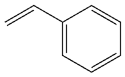 | 10 | 10 | 24 | 73 | 83:11:6 |
| 46 | 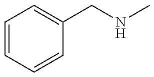 | 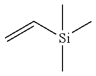 | 10 | 10 | 24 | 61 | — |
| 47 | 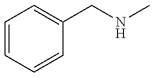 | 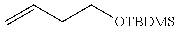 | 10 | 10 | 24 | 99 | — |
| 48 | 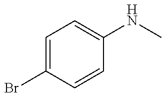 | 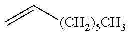 | 5 | 5 | 2 | 96 | 99:1 |
| 49 | 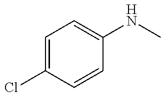 | 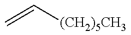 | 5 | 5 | 2 | 97 | 99:1 |

ªReaction conditions: amine (0.5 mmol), alkene (0.75 mmol).
^{b}Neat reaction conditions: amine (0.5 mmol), alkene (2.5 mmol), no solvent.
^{c}Reaction conditions: amine (0.5 mmol), alkene (0.6 mmol).
*Branched: Linear For example (Entry 1):

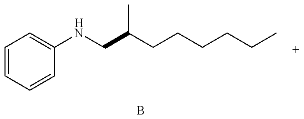

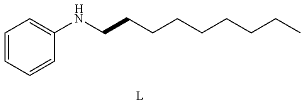

Reaction Vessel Comparison. Titanium catalysts based on commercially available Ti(NMe₂)₄ (Ti1) are known to be extremely susceptible to side reactions and inhibitions in the catalytic turnovers due to the release of dimethylamine.⁴¹ Dimethylamine can inhibit the formation of titanaaziridines, the catalytic active species for the hydroaminoalkylation⁴², and consequently inhibits the alkene insertion step in the catalytic cycle. This “unwanted” side reaction can be so prevalent that industrial relevant dimethylamine was already reported as substrate for hydroaminoalkylation.⁴⁰ To achieve high reactivities for this type of catalysts the generated gaseous dimethylamine typically has to be released from the reaction system.

However, surprisingly, during the present reactivity studies, the catalyst system based on Ti1 and L7 did not show any indication of interference, even though the reactions were carried out in relatively small J. Young NMR tubes with a trivial amount of headspace. Consequently, a variety of different reaction vessels were tested with different volumes of headspace to confirm the neglectable relevance of dimethylamine for the activity of this catalyst system. For this, the benchmark reaction of N-methylaniline and 1-octene with 5% catalyst loading was used and the reactions scaled up slightly to 1.0 mmol of amine. Additionally, toluene was not used as solvent. Instead, neat reaction conditions were used with 2.5 eq. of alkene as neat conditions may, for example, improve the catalyst activity and are advantageous in applications in the field of green chemistry.

First, commercially available high-pressure glass vials were tested, which are widely accepted by organic chemists and are normally used for microwave reactors. Under these conditions a drop in conversion (64% after 2 h) in comparison to the NMR screening technique (95%) with toluene as solvent was observed. However, this decrease in reactivity was considerably lower than for other reported systems⁴⁰ and full conversion was achieved after 4 h. Surprisingly, by switching to significantly larger reaction vessels (100 mL Schlenk flask) no increase in reactivity was observed (full conversion after 4 h) and only the implementation of another pressure relief in form of a balloon could reduce the reaction time slightly to 3 h. Motivated by these findings, we explored procedures for hydroaminoalkylation without the necessity of glove box techniques. For this, the entire reaction was still carried out with dry reagents and under exclusion of oxygen and moisture but was outside of the glovebox using Teflon caped vials, standard Schlenk line techniques and balloons filled with inert gas, which are generally well-known techniques for pure organic chemists. Surprisingly, with this procedure, not only were the same conversion numbers achieved, but also due to practical handling of all substrates before and during the reaction, this method has advantages to this end over previously reported synthetic procedures for hydroaminoalkylation.

Synthetic Application. Catalyst systems like Ti1+L7 with its easy-to-handle synthetic procedures, may be of interest as a catalyst for a variety of different synthetic applications, for example, in the field of monomer design for nitrogen-containing polymers. N-methylaniline was accordingly reacted with norbornadiene (NBD) and 1,5-cyclooctadiene (COD) neat to generate amine-containing monomers 1 and 2 which may be subsequently used in ROMP. Both reactions are completed quantitatively within 8 h for NBD and 72 h for COD. While not wishing to be limited by theory, the relatively long reaction time for the COD reaction is attributed to the large ring size, which likely inhibits the insertion step of the alkene in the catalytic cycle, and the low ring strain of the cyclic alkene. Because of the neat conditions and the quantitative yields, both monomers can simply be isolated in high purity by filtration of the catalyst and removal of the residual diene in vacuo. No further purification steps were needed and the used diene can be recycled. Consequently, this synthetic concept for N-containing monomers 1 and 2 is very efficient and especially “green” due to the use of earth-abundant titanium catalysts and no requirement for additional solvents.

Furthermore, the synthesis of building blocks for medicinally relevant small molecules may also be of interest. To investigate the access to these product classes, we reacted 4-methoxy-N-methylaniline and alkenes bearing silyl protected alcohols, which showed high reactivity for hydroaminoalkylation. With the isolation of this hydroaminoalkylation product 3 in excellent yields (94%), two different pathways are potentially open for further functionalization. First, we attempted to deprotect and oxidize 3 in one step using periodic acid with via a known procedure⁴³ to generate the corresponding β-substituted γ-amino acid, an interesting substance class to target specific proteins with representatives such as pregabalin and gabapentin, which are used as anticonvulsants.⁴⁴ This product was not isolated, due to issues with overoxidation and the high solubility in water, however it could be identified as a minor product via LC-MS analysis (12%). A stepwise approach was then used to synthesize an isolable protected amino acid with deprotection of the alcohol function with TBAF 4 BOC-protection of the amine and finally direct oxidation of the alcohol to the amino acid 5 using KMnO₄ with an overall good yield of 77%.

Another way to further transform the silyl protected amino alcohols is by cyclization using p-toluenesulfonyl fluoride 6 and subsequent deprotection of the p-methoxyphenyl ether (PMP) group. 3-Methylpyrrolidines are an interesting compound class as a target, because they can be used as building blocks in medicinal chemistry, e.g. for kinase inhibitors and small-molecule chemical probes for proteins.⁴⁵ Furthermore, β-methylated amine products are desirable, because of their “magic methyl” effects in medicinal chemistry.⁴⁶ For the PMP-deprotection three different oxidation reagents were tested: ceric ammonium nitrate (CAN), periodic acid and trichloroisocyanuric acid (TCCA). Whereas CAN and periodic acid did not facilitate the corresponding cyclic secondary amine in good yields, TCCA generated the amine hydrochloride 7 with an overall yield of 62%. The synthetic costs (neglecting work time and solvents) were calculated and even though the target molecule loses 62% of molecular mass, the price was still around one magnitude cheaper than from commercial sources, due to the use of low-cost titanium catalyzed hydroaminoalkylation. Additionally, this shows the value of using PMP-protected amines for hydroaminoalkylation, which exhibit in general very high reactivity for this transformation.

Hydroaminoalkylation of polymers comprising alkene moieties. The titanium-catalyzed hydroaminoalkylation of exemplary atactic vinyl-terminated polypropylenes was also carried out using the catalyst system based on Ti1 and L7. The reaction was carried out in toluene-d₈ at 160° C. in the presence of 33 mol % trimethoxybenzene (TMB) using the conditions summarized in Table 7. A scaled-up reaction was also carried out in less than 0.5 mL toluene using a catalyst loading of 10 mol % Ti for 3-7 days using the reagents summarized in Table 8. Additional scaled up reactions were carried out similar to that described for the reagents of Table 8 substantially in the absence of solvent; only the catalyst itself was dissolved in a minimum of toluene. The amount of toluene used to dissolve the catalyst varies with the amount of catalyst.

TABLE 7
Titanium catalyzed hydroaminoalkylation of vinyl-terminated polypropylenes.

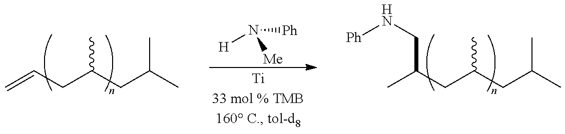

| Mn (g/mol) | Amine | Time (h) | Catalyst Loading |
|---|---|---|---|
| 347 | N-methylaniline | 10 | 5 |
| 842 | N-methylaniline | 10 | 5 |
| 8500 | N-methylaniline | 120 | 20 |

TABLE 8
Titanium catalyzed hydroaminoalkylation of vinyl-terminated polypropylenes.

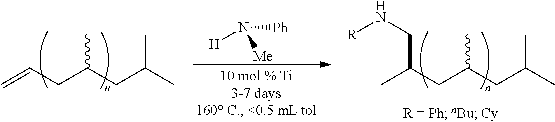

| Mn (g/mol) | Amine | Mass Loading (g) |
|---|---|---|
| 347 | N-methylaniline | 1 |
| 347 | N-methylcyclohexylamine | 1 |
| 842 | N-methylaniline | 5 |
| 842 | N-methylcyclohexylamine | 5 |
| 842 | N-methylbutan-1-amine | 5 |

As monitored by NMR spectroscopy, complete reactivity was observed by the disappearance of the alkene signals. The resulting materials can be isolated on gram scale and modified materials properties result in changed T_{g}s.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION

¹ Franke, R.; Selent, D.; Börner, A., Applied Hydroformylation. Chemical Reviews 2012, 112 (11), 5675-5732.
² Edwards, P. M.; Schafer, L. L., Early transition metal-catalyzed C—H alkylation: hydroaminoalkylation for Csp3-Csp3 bond formation in the synthesis of selectively substituted amines. Chemical Communications 2018, 54 (89), 12543-12560.
³ Vitaku, E.; Smith, D. T.; Njardarson, J. T., Analysis of the Structural Diversity, Substitution Patterns, and Frequency of Nitrogen Heterocycles among U.S. FDA Approved Pharmaceuticals. Journal of Medicinal Chemistry 2014, 57 (24), 10257-10274.
⁴ Clerici, M. G.; Maspero, F., Catalytic C-Alkylation of Secondary Amines with Alkenes. Synthesis 1980, 1980 (04), 305-306.
⁵ Herzon, S. B.; Hartwig, J. F., Direct, Catalytic Hydroaminoalkylation of Unactivated Olefins with N-Alkyl Arylamines. Journal of the American Chemical Society 2007, 129 (21), 6690-6691.
⁶ (a) Manßen, M.; Schafer, L. L., Titanium catalysis for the synthesis of fine chemicals—development and trends. Chem Soc Rev 2020, 49 (19), 6947-6994; (b) Schafer, L. L.; Manßen, M.; Edwards, P. M.; Lui, E. K. J.; Griffin, S. E.; Dunbar, C. R., Chapter Seven—Titanium catalyzed synthesis of amines and N-heterocycles. In Advances in Organometallic Chemistry, Pérez, P. J., Ed. Academic Press: 2020; Vol. 74, pp 405-468; (c) Garcia, P.; Lau, Y. Y.; Perry, M. R.; Schafer, L. L., Phosphoramidate Tantalum Complexes for Room-Temperature C—H Functionalization: Hydroaminoalkylation Catalysis. Angewandte Chemie International Edition 2013, 52 (35), 9144-9148; (d) Chong, E.; Brandt, J. W.; Schafer, L. L., 2-Pyridonate Tantalum Complexes for the Intermolecular Hydroaminoalkylation of Sterically Demanding Alkenes. Journal of the American Chemical Society 2014, 136 (31), 10898-10901.
⁷ (a) Hunt, A. J.; Farmer, T. J.; Clark, J. H., Elemental Sustainability and the Importance of Scarce Element Recovery. In Element Recovery and Sustainability, The Royal Society of Chemistry: 2013; pp 1-28; (b) Haynes, W. M.; Lide, D. R.; Bruno, T. J., CRC handbook of chemistry and physics. 2017.
⁸ Collins, R. A.; Russell, A. F.; Mountford, P., Group 4 metal complexes for homogeneous olefin polymerisation: a short tutorial review. Applied Petrochemical Research 2015, 5 (3), 153-171.
⁹ Kubiak, R.; Prochnow, I.; Doye, S., Titanium-Catalyzed Hydroaminoalkylation of Alkenes by C—H Bond Activation at sp3 Centers in the α-Position to a Nitrogen Atom. Angewandte Chemie International Edition 2009, 48 (6), 1153-1156.
¹⁰ Prochnow, I.; Kubiak, R.; Frey, O. N.; Beckhaus, R.; Doye, S., Tetrabenzyltitanium: An Improved Catalyst for the Activation of sp3 C—H Bonds Adjacent to Nitrogen Atoms. ChemCatChem 2009, 1 (1), 162-172.
¹¹ Dörfler, J.; Doye, S., A Commercially Available Tantalum Catalyst for the Highly Regioselective Intermolecular Hydroaminoalkylation of Styrenes. European Journal of Organic Chemistry 2014, 2014 (13), 2790-2797.
¹² (a) Kubiak, R.; Prochnow, I.; Doye, S., [Ind₂TiMe₂]: A Catalyst for the Hydroaminomethylation of Alkenes and Styrenes. Angewandte Chemie International Edition 2010, 49 (14), 2626-2629; (b) Rohjans, S. H.; Ross, J. H.; Lühning, L. H.; Sklorz, L.; Schmidtmann, M.; Doye, S., Titanium Catalysts with Linked Indenyl-Amido Ligands for Hydroamination and Hydroaminoalkylation Reactions. Organometallics 2018, 37 (23), 4350-4357; (c) Preuß, T.; Saak, W.; Doye, S., Titanium-Catalyzed Intermolecular Hydroaminoalkylation of Conjugated Dienes. Chemistry—A European Journal 2013, 19 (12), 3833-3837.
¹³ (a) Leitch, D. C.; Payne, P. R.; Dunbar, C. R.; Schafer, L. L., Broadening the Scope of Group 4 Hydroamination Catalysis Using a Tethered Ureate Ligand. Journal of the American Chemical Society 2009, 131 (51), 18246-18247; (b) Leitch, D. C.; Platel, R. H.; Schafer, L. L., Mechanistic Elucidation of Intramolecular Aminoalkene Hydroamination Catalyzed by a Tethered Bis(ureate) Complex: Evidence for Proton-Assisted C—N Bond Formation at Zirconium. Journal of the American Chemical Society 2011, 133 (39), 15453-15463; (c) Wood, M. C.; Leitch, D. C.; Yeung, C. S.; Kozak, J. A.; Schafer, L. L., Chiral Neutral Zirconium Amidate Complexes for the Asymmetric Hydroamination of Alkenes. Angewandte Chemie International Edition 2007, 46 (3), 354-358; (d) Bexrud, J. A.; Schafer, L. L., Zirconium bis(pyridonate): a modified amidate complex for enhanced substrate scope in aminoalkene cyclohydroamination. Dalton Transactions 2010, 39 (2), 361-363; (e) Braun, C.; Nieger, M.; Bräse, S.; Schafer, L. L., Planar-Chiral [2.2]Paracyclophane-Based Pyridonates as Ligands for Tantalum-Catalyzed Hydroaminoalkylation. ChemCatChem 2019, 11 (21), 5264-5268; (f) Chong, E.; Schafer, L. L., 2-Pyridonate Titanium Complexes for Chemoselectivity. Accessing Intramolecular Hydroaminoalkylation over Hydroamination. Organic Letters 2013, 15 (23), 6002-6005; (g) Brandt, J. W.; Chong, E.; Schafer, L. L., Ligand Effects and Kinetic Investigations of Sterically Accessible 2-Pyridonate Tantalum Complexes for Hydroaminoalkylation. ACS Catalysis 2017, 7 (9), 6323-6330.
¹⁴ (a) Kuanr, N.; Tomkovic, T.; Gilmour, D. J.; Perry, M. R.; Hsiang, S. J.; van Ruymbeke, E.; Hatzikiriakos, S. G.; Schafer, L. L., Dynamic Cross-Linking of Catalytically Synthesized Poly(Aminonorbomenes). Macromolecules 2020, 53 (7), 2649-2661; (b) Perry, M. R.; Ebrahimi, T.; Morgan, E.; Edwards, P. M.; Hatzikiriakos, S. G.; Schafer, L. L., Catalytic Synthesis of Secondary Amine-Containing Polymers: Variable Hydrogen Bonding for Tunable Rheological Properties. Macromolecules 2016, 49 (12), 4423-4430.
¹⁵ Elkin, T.; Kulkarni, N. V.; Tumanskii, B.; Botoshansky, M.; Shimon, L. J. W.; Eisen, M. S., Synthesis and Structure of Group 4 Symmetric Amidinate Complexes and Their Reactivity in the Polymerization of α-Olefins. Organometallics 2013, 32 (21), 6337-6352.
¹⁶ Dörfler, J.; Bytyqi, B.; Huller, S.; Mann, N. M.; Brahms, C.; Schmidtmann, M.; Doye, S., An Aminopyridinato Titanium Catalyst for the Intramolecular Hydroaminoalkylation of Secondary Aminoalkenes. Advanced Synthesis & Catalysis 2015, 357 (10), 2265-2276.
¹⁷ (a) Dörfler, J.; Doye, S., Aminopyridinato Titanium Catalysts for the Hydroaminoalkylation of Alkenes and Styrenes. Angewandte Chemie International Edition 2013, 52 (6), 1806-1809; (b) Dörfler, J.; Preuß, T.; Schischko, A.; Schmidtmann, M.; Doye, S., A 2,6-Bis(phenylamino)pyridinato Titanium Catalyst for the Highly Regioselective Hydroaminoalkylation of Styrenes and 1,3-Butadienes. Angewandte Chemie International Edition 2014, 53 (30), 7918-7922; (c) Warsitz, M.; Doye, S., Two-Step Procedure for the Synthesis of 1,2,3,4-Tetrahydroquinolines. European Journal of Organic Chemistry https://doi.org/10.1002/ejoc.202001337; (d) Warsitz, M.; Doye, S., Linear Hydroaminoalkylation Products from Alkyl-Substituted Alkenes. Chemistry—A European Journal https://doi.org/10.1002/chem.202003223.
¹⁸ Bielefeld, J.; Doye, S., Dimethylamine as a Substrate in Hydroaminoalkylation Reactions. Angewandte Chemie International Edition 2017, 56 (47), 15155-15158.
¹⁹ Rosien, M.; Toben, I.; Schmidtmann, M.; Beckhaus, R.; Doye, S., Titanium-Catalyzed Hydroaminoalkylation of Ethylene. Chemistry 2020, 26 (10), 2138-2142.
²⁰ Bielefeld, J.; Doye, S., Fast Titanium-Catalyzed Hydroaminomethylation of Alkenes and the Formal Conversion of Methylamine. Angew Chem Int Ed Engl 2020, 59 (15), 6138-6143.
²¹ Vilaivan, T., A rate enhancement of tert-butoxycarbonylation of aromatic amines with Boc2O in alcoholic solvents. Tetrahedron Letters 2006, 47 (38), 6739-6742.
²² Matos, P. M.; Lewis, W.; Moore, J. C.; Stockman, R. A., Sulfonimidates: Useful Synthetic Intermediates for Sulfoximine Synthesis via C—S Bond Formation. Organic Letters 2018, 20 (12), 3674-3677.
²³ Eisenberger, P.; Ayinla, R. O.; Lauzon, J. M. P.; Schafer, L. L., Tantalum-Amidate Complexes for the Hydroaminoalkylation of Secondary Amines: Enhanced Substrate Scope and Enantioselective Chiral Amine Synthesis. Angewandte Chemie International Edition 2009, 48 (44), 8361-8365.
²⁴ DiPucchio, R. C.; Rosca, S. C.; Athavan, G.; Schafer, L. L., Exploiting Natural Complexity: Synthetic Terpenoid-Alkaloids by Regioselective and Diastereoselective Hydroaminoalkylation Catalysis. ChemCatChem 2019, 11 (16), 3871-3876.
²⁵ Majer, P.; Randad, R. S., A Safe and Efficient Method for Preparation of N,N′-Unsymmetrically Disubstituted Ureas Utilizing Triphosgene. The Journal of Organic Chemistry 1994, 59 (7), 1937-1938.
²⁶ Jin, G.; Jones, C.; Junk, P. C.; Lippert, K. A.; Rose, R. P.; Stasch, A., Synthesis and characterisation of bulky guanidines and phosphaguanidines: precursors for low oxidation state metallacycles. New Journal of Chemistry 2009, 33 (1), 64-75.
²⁷ McAlexander, L. H.; Li, L.; Yang, Y.; Pollitte, J. L.; Xue, Z., Syntheses of Titanium and Zirconium Chlorotrialkyl Complexes and Crystal Structure of Chlorotrineopentylzirconium Containing Unusual Linear Symmetric Metal-Chloride Polymeric Chains. Inorganic Chemistry 1998, 37 (6), 1423-1426.
²⁸ Kuanr, N.; Tomkovic, T.; Gilmour, D. J.; Perry, M. R.; Hsiang, S. J.; van Ruymbeke, E.; Hatzikiriakos, S. G.; Schafer, L. L., Dynamic Cross-Linking of Catalytically Synthesized Poly(Aminonorbomenes). Macromolecules 2020, 53 (7), 2649-2661.
²⁹ Payne, P. R.; Garcia, P.; Eisenberger, P.; Yim, J. C. H.; Schafer, L. L., Tantalum Catalyzed Hydroaminoalkylation for the Synthesis of α- and β-Substituted N-Heterocycles. Organic Letters 2013, 15 (9), 2182-2185.
³⁰ Kordes, M.; Winsel, H.; de Meijere, A., A New Short Access to Amino Acids Incorporating an Aminocyclopropyl Moiety from N,N-Dibenzylcarboxamides. European Journal of Organic Chemistry 2000, 2000 (18), 3235-3245.
³¹ Edwards, P. M.; Schafer, L. L., In Situ Generation of a Regio- and Diastereoselective Hydroaminoalkylation Catalyst Using Commercially Available Starting Materials. Organic Letters 2017, 19 (21), 5720-5723.
³² Lin, T. Y.; Kingsbury, C. A.; Cromwell, N. H., The synthesis of 4-methyl-2-pyrrolidones and 3-methyl-1-pyrrolidines and their mass spectral study. Journal of Heterocyclic Chemistry 1984, 21 (6), 1871-1875.
³³ McAlexander, L. H.; Li, L.; Yang, Y.; Pollitte, J. L.; Xue, Z., Syntheses of Titanium and Zirconium Chlorotrialkyl Complexes and Crystal Structure of Chlorotrineopentylzirconium Containing Unusual Linear Symmetric Metal-Chloride Polymeric Chains. Inorganic Chemistry 1998, 37 (6), 1423-1426.
³⁴ Prochnow, I.; Kubiak, R.; Frey, O. N.; Beckhaus, R.; Doye, S., Tetrabenzyltitanium: An Improved Catalyst for the Activation of sp³ C—H Bonds Adjacent to Nitrogen Atoms. ChemCatChem 2009, 1 (1), 162-172.
³⁵ Brandt, J. W.; Chong, E.; Schafer, L. L., Ligand Effects and Kinetic Investigations of Sterically Accessible 2-Pyridonate Tantalum Complexes for Hydroaminoalkylation. ACS Catalysis 2017, 7 (9), 6323-6330.
³⁶ Chong, E.; Schafer, L. L., 2-Pyridonate Titanium Complexes for Chemoselectivity. Accessing Intramolecular Hydroaminoalkylation over Hydroamination. Organic Letters 2013, 15 (23), 6002-6005.
³⁷ Robak, M. T.; Herbage, M. A.; Ellman, J. A., Synthesis and Applications of tert-Butanesulfinamide. Chemical Reviews 2010, 110 (6), 3600-3740.
³⁸ (a) Dörfler, J.; Bytyqi, B.; Huller, S.; Mann, N. M.; Brahms, C.; Schmidtmann, M.; Doye, S., An Aminopyridinato Titanium Catalyst for the Intramolecular Hydroaminoalkylation of Secondary Aminoalkenes. Advanced Synthesis & Catalysis 2015, 357 (10), 2265-2276; (b) Dörfler, J.; Doye, S., Aminopyridinato Titanium Catalysts for the Hydroaminoalkylation of Alkenes and Styrenes. Angewandte Chemie International Edition 2013, 52 (6), 1806-1809; (c) Dörfler, J.; Preuß, T.; Schischko, A.; Schmidtmann, M.; Doye, S., A 2,6-Bis(phenylamino)pyridinato Titanium Catalyst for the Highly Regioselective Hydroaminoalkylation of Styrenes and 1,3-Butadienes. Angewandte Chemie International Edition 2014, 53 (30), 7918-7922; (d) Warsitz, M.; Doye, S., Two-Step Procedure for the Synthesis of 1,2,3,4-Tetrahydroquinolines. European Journal of Organic Chemistry https://doi.org/10.1002/ejoc.202001337; (e) Warsitz, M.; Doye, S., Linear Hydroaminoalkylation Products from Alkyl-Substituted Alkenes. Chemistry—A European Journal https://doi.org/10.1002/chem.202003223; (f) Dörfler, J.; Preuß, T.; Brahms, C.; Scheuer, D.; Doye, S., Intermolecular hydroaminoalkylation of alkenes and dienes using a titanium mono(formamidinate) catalyst. Dalton Transactions 2015, 44 (27), 12149-12168; (g) Bielefeld, J.; Doye, S., Dimethylamine as a Substrate in Hydroaminoalkylation Reactions. Angewandte Chemie International Edition 2017, 56 (47), 15155-15158; (h) Rosien, M.; Toben, I.; Schmidtmann, M.; Beckhaus, R.; Doye, S., Titanium-Catalyzed Hydroaminoalkylation of Ethylene. Chemistry 2020, 26 (10), 2138-2142.
³⁹ (a) Dörfler, J.; Doye, S., Aminopyridinato Titanium Catalysts for the Hydroaminoalkylation of Alkenes and Styrenes. Angewandte Chemie International Edition 2013, 52 (6), 1806-1809; (b) Dörfler, J.; Preuß, T.; Schischko, A.; Schmidtmann, M.; Doye, S., A 2,6-Bis(phenylamino)pyridinato Titanium Catalyst for the Highly Regioselective Hydroaminoalkylation of Styrenes and 1,3-Butadienes. Angewandte Chemie International Edition 2014, 53 (30), 7918-7922; (c) Dörfler, J.; Preuß, T.; Brahms, C.; Scheuer, D.; Doye, S., Intermolecular hydroaminoalkylation of alkenes and dienes using a titanium mono(formamidinate) catalyst. Dalton Transactions 2015, 44 (27), 12149-12168; (d) Bielefeld, J.; Kurochkina, E.; Schmidtmann, M.; Doye, S., New Titanium Complexes and Their Use in Hydroamination and Hydroaminoalkylation Reactions. European Journal of Inorganic Chemistry 2019, 2019 (32), 3713-3718.
⁴⁰ Bielefeld, J.; Doye, S., Fast Titanium-Catalyzed Hydroaminomethylation of Alkenes and the Formal Conversion of Methylamine. Angew Chem Int Ed Engl 2020, 59 (15), 6138-6143.
⁴¹ Bielefeld, J.; Doye, S., Fast Titanium-Catalyzed Hydroaminomethylation of Alkenes and the Formal Conversion of Methylamine. Angew Chem Int Ed Engl 2020, 59 (15), 6138-6143.
⁴² (a) Manßen, M.; Lauterbach, N.; Dörfler, J.; Schmidtmann, M.; Saak, W.; Doye, S.; Beckhaus, R., Efficient Access to Titanaaziridines by C—H Activation of N-Methylanilines at Ambient Temperature. Angewandte Chemie International Edition 2015, 54 (14), 4383-4387; (b) Prochnow, I.; Zark, P.; Müller, T.; Doye, S., The Mechanism of the Titanium-Catalyzed Hydroaminoalkylation of Alkenes. Angewandte Chemie International Edition 2011, 50 (28), 6401-6405.
⁴³ Verkade, J. M. M.; van Hemert, L. J. C.; Quaedflieg, P. J. L. M.; Alsters, P. L.; van Delft, F. L.; Rutjes, F. P. J. T., Mild and efficient deprotection of the amine protecting p-methoxyphenyl (PMP) group. Tetrahedron Letters 2006, 47 (46), 8109-8113.
⁴⁴ Belliotti, T. R.; Capiris, T.; Ekhato, I. V.; Kinsora, J. J.; Field, M. J.; Heffner, T. G.; Meltzer, L. T.; Schwarz, J. B.; Taylor, C. P.; Thorpe, A. J.; Vartanian, M. G.; Wise, L. D.; Zhi-Su, T.; Weber, M. L.; Wustrow, D. J., Structure-Activity Relationships of Pregabalin and Analogues That Target the +2-δ Protein. Journal of Medicinal Chemistry 2005, 48 (7), 2294-2307.
⁴⁵ (a) Noji, S.; Hara, Y.; Miura, T.; Yamanaka, H.; Maeda, K.; Hori, A.; Yamamoto, H.; Obika, S.; Inoue, M.; Hase, Y.; Orita, T.; Doi, S.; Adachi, T.; Tanimoto, A.; Oki, C.; Kimoto, Y.; Ogawa, Y.; Negoro, T.; Hashimoto, H.; Shiozaki, M., Discovery of a Janus Kinase Inhibitor Bearing a Highly Three-Dimensional Spiro Scaffold: JTE-052 (Delgocitinib) as a New Dermatological Agent to Treat Inflammatory Skin Disorders. Journal of Medicinal Chemistry 2020, 63 (13), 7163-7185; (b) Moustakim, M.; Christott, T.; Monteiro, O. P.; Bennett, J.; Giroud, C.; Ward, J.; Rogers, C. M.; Smith, P.; Panagakou, I.; Díaz-Sáez, L.; Felce, S. L.; Gamble, V.; Gileadi, C.; Halidi, N.; Heidenreich, D.; Chaikuad, A.; Knapp, S.; Huber, K. V. M.; Farie, G.; Heer, J.; Manevski, N.; Poda, G.; Al-awar, R.; Dixon, D. J.; Brennan, P. E.; Fedorov, O., Discovery of an MLLT1/3 YEATS Domain Chemical Probe. Angewandte Chemie International Edition 2018, 57 (50), 16302-16307.
⁴⁶ Barreiro, E. J.; Kümmerle, A. E.; Fraga, C. A. M., The Methylation Effect in Medicinal Chemistry. Chemical Reviews 2011,111 (9), 5215-5246.

What is claimed is:

1. A method for the hydroaminoalkylation of an olefin, the method comprising reacting the olefin with a secondary amine in the presence of a catalyst of Formula (I):
LₓM(R¹)_{y}(I),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L is a ligand of Formula (II) 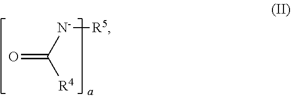
wherein
each a is independently 1 or 2;
each R⁴ is independently C₁₋₄₀alkyl, C₃₋₄₀cycloalkyl, substituted or unsubstituted aryl or NR⁶R⁷;
(i) when a is 1,
R⁵ is C₁₋₄₀alkyl, C₃₋₄₀cycloalkyl or substituted or unsubstituted aryl; and
R⁶ and R⁷ are each independently C₁₋₄alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and
R⁶ is C₁₋₄₀alkyl, C₃₋₄₀cycloalkyl, substituted or unsubstituted aryl or C₁₋₁₀alkylene-aryl, wherein the aryl is substituted or unsubstituted;
(ii) when a is 2,
R⁵ is C₁₋₄₀alkylene, C₃₋₄₀cycloalkylene or substituted or unsubstituted arylene; and R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

2. The method of claim 1, wherein a is 1.

3. The method of claim 1 or 2, wherein x is 1 or 2.

4. The method of claim 3, wherein x is 1.

5. The method of any one of claims 2 to 4, wherein R⁵ is C₁₋₁₆alkyl, C₃₋₁₆cycloalkyl or substituted aryl.

6. The method of claim 5, wherein R⁵ is substituted aryl.

7. The method of claim 6, wherein R⁵ is 2,6-diisopropylphenyl.

8. The method of any one of claims 2 to 7, wherein R⁴ is NR⁶R⁷, and R⁶ and R⁷ are each independently C₁₋₁₆alkyl, C₃₋₁₆cycloalkyl, substituted or unsubstituted aryl or C₁₋₆alkylene-aryl.

9. The method of claim 8, wherein R⁶ and R⁷ are different.

10. The method of claim 8 or 9, wherein R⁶ is C₁₋₆alkyl and R⁷ is C₁₋₄alkylene-aryl.

11. The method of claim 10, wherein R⁶ is methyl and R⁷ is —CH(CH₃)-phenyl.

12. The method of any one of claims 2 to 7, wherein R⁴ is C₁₋₁₀alkyl or unsubstituted aryl.

13. The method of claim 12, wherein R⁴ is tert-butyl or phenyl.

14. The method of any one of claims 1 to 13, wherein R¹ is NMe₂ or CH₂Si(CH₃)₃.

15. The method of claim 14, wherein R¹ is NMe₂.

16. The method of any one of claims 1 to 15, wherein M is Ti.

17. The method of any one of claims 1 to 16, wherein the catalyst is prepared from the reaction of a compound of Formula (III) with a group 4 metal complex of Formula (IV):

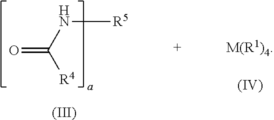

18. The method of any one of claims 1 to 17, wherein the catalyst is generated in situ.

19. The method of any one of claims 1 to 18, wherein the reaction of the olefin with the secondary amine is carried out without addition of solvent.

20. The method of any one of claims 1 to 19, wherein the olefin comprises a terminal alkene.

21. The method of any one of claims 1 to 20, wherein the olefin comprises a silyl protected alcohol.

22. The method of any one of claims 1 to 19, wherein the olefin comprises a cyclic alkene.

23. The method of claim 22, wherein the reaction of the olefin with the secondary amine produces an amine-substituted monomer for ring-opening metathesis polymerization (ROMP).

24. The method of any one of claims 1 to 19, wherein the olefin is a polymer comprising an alkene.

25. The method of claim 24, wherein the polymer is an alkene-terminated polyolefin.

26. The method of any one of claims 1 to 25, wherein the secondary amine is of the formula NR^{a}R^{b}, wherein R^{a} and R^{b} are each independently substituted or unsubstituted C₁₋₄₀alkyl, C₃₋₄₀cycloalkyl, C₁₋₁₀alkylene-C₃₋₄₀cycloalkyl, aryl or C₁₋₁₀alkylene-aryl or R^{a} and R^{b} are bonded together, thereby forming, together with the nitrogen atom they are both bound to, a substituted or unsubstituted heterocycle that is optionally part of a condensed ring system.

27. The method of claim 26, wherein R^{a} is C₁₋₄alkyl and R^{b} is substituted or unsubstituted aryl.

28. The method of claim 27, wherein the amine is of the formula 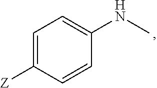
wherein Z is H, halo, —O—C₁₋₁₀alkyl, —O—C₃₋₁₀cycloalkyl, C₁₋₁₀alkyl or C₃₋₁₀cycloalkyl.

29. The method of any one of claims 1 to 19, wherein the hydroaminoalkylation is intramolecular.

30. A compound of Formula I(a):
(L^{a})ₓM(R¹)_{y}I(a),
wherein
x is 1 to 4 and y is 0 to 3, wherein the sum of x and y is 4;
M is a group 4 metal;
R¹ is N(R²)₂, —C₁₋₆alkylene-Si(R³)₃ or —C₁₋₆alkylene-aryl;
R² and R³ are each independently C₁₋₆alkyl; and
L^{a} is a ligand of Formula II(a) 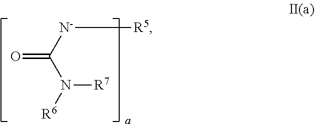
wherein
each a is independently 1 or 2;
(i) when a is 1,
R⁵ is C₁₋₄₀alkyl, C₃₋₄₀cycloalkyl or substituted or unsubstituted aryl; and
R⁶ and R⁷ are each independently C₁₋₄alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle; or R⁵ and R⁷ are bonded together, thereby forming, together with the nitrogen atom they are both bound to, a substituted or unsubstituted heterocycle; and
R⁶ is C₁₋₄₀alkyl, C₃₋₄₀cycloalkyl, substituted or unsubstituted aryl or C₁₋₁₀alkylene-aryl, wherein the aryl is substituted or unsubstituted;
(ii) when a is 2,
R⁵ is C₁₋₄₀alkylene, C₃₋₄₀cycloalkylene or substituted or unsubstituted arylene; and R⁶ and R⁷ are each independently C₁₋₄₀alkyl, C₃₋₄₀cycloalkyl, substituted or unsubstituted aryl or C₁₋₁₀alkylene-aryl, wherein the aryl is substituted or unsubstituted; or R⁶ and R⁷ are bonded together, thereby forming, together with the nitrogen atom they are both bound to, a substituted or unsubstituted heterocycle.

31. The compound of claim 30, wherein a is 1.

32. The compound of claim 30 or 31, wherein x is 1 or 2.

33. The compound of claim 32, wherein x is 1.

34. The compound of any one of claims 31 to 33, wherein R⁵ is C₁₋₁₆alkyl, C₃₋₁₆cycloalkyl or substituted aryl.

35. The compound of claim 34, wherein R⁵ is substituted aryl.

36. The compound of claim 35, wherein R⁵ is 2,6-diisopropylphenyl.

37. The compound of any one of claims 31 to 36, wherein R⁶ and R⁷ are each independently C₁₋₁₆alkyl, C₃₋₁₆cycloalkyl, substituted or unsubstituted aryl or C₁₋₆alkylene-aryl.

38. The compound of claim 37, wherein R⁶ and R⁷ are different.

39. The compound of claim 38, wherein R⁶ is C₁₋₆alkyl and R⁷ is C₁₋₄alkylene-aryl.

40. The compound of claim 39, wherein R⁶ is methyl and R⁷ is —CH(CH₃)-phenyl.

41. The compound of any one of claims 30 to 40, wherein R¹ is NMe₂ or CH₂Si(CH₃)₃.

42. The compound of claim 41, wherein R₁ is NMe₂.

43. The compound of any one of claims 30 to 42, wherein M is Ti.
